(19) United States
(12) Patent Application Publication
(10) Pub. No.: US 2013/0330412 A1
(43) Pub. Date: Dec. 12, 2013

(54) SMART POLYMERIC NANOPARTICLES WHICH OVERCOME MULTIDRUG RESISTANCE TO CANCER THERAPEUTICS AND TREATMENT-RELATED SYSTEMIC TOXICITY

(71) Applicants: Anirban Maitra, Baltimore, MD (US); Dipankar Pramanik, Baltimore, MD (US)

(72) Inventors: Anirban Maitra, Baltimore, MD (US); Dipankar Pramanik, Baltimore, MD (US)

(73) Assignee: THE JOHNS HOPKINS UNIVERSITY, Baltimore, MD (US)

(21) Appl. No.: 13/992,777

(22) PCT Filed: Dec. 8, 2011
(86) PCT No.: PCT/US11/63870

§ 371 Date: Aug. 19, 2013

Related U.S. Application Data
(60) Provisional application No. 61/421,709, filed on Dec. 10, 2010.

(51) Int. Cl. A61K 9/51; A61K 45/06; A61K 31/704; A61K 31/12

(52) U.S. Cl. CPC A61K 9/5138; A61K 31/12; A61K 45/06; A61K 31/704
USPC 424/497; 514/679; 514/34

(57) ABSTRACT

Polymeric nanoparticles with a hydrophobic core that encapsulates curcumin and a hydrophilic shell with one or more chemotherapeutic agents (e.g., doxorubicin) associated with the shell surface are formed from N-isopropylacryl amide (NEPAAM), acrylic acid (AA), and at least one vinyl monomer selected from the group consisting of vinyl acetate, 4-vinyl benzoic acid, methylmethacrylate, vinylmethacrylate, N-vinylpyrrolidone, N-vinyl piperidone, N-vinyl caprolacum, N-vinyl carbazole, and styrene, where the NIPAAM, the AA, and the vinyl monomer are present at molar ratios of 50-70:10-30:10-30 for NIPAAM:AA:vinyl monomer. These nanoparticles effectively overcome multidrug resistance and ameliorate cardiomyopathy in vivo.

### FIELD OF THE INVENTION

The invention relates to nanoparticle compositions for solubilization and encapsulation of medicines, including medicines that are poorly water-soluble, and particularly including curcumin in combination with at least one cancer therapeutic (e.g., anthracyclines (doxorubicin, daunorubicin), paclitaxel and other taxanes, cisplatin and other platinum compounds, topoisomerase inhibitors, etc.). More particularly, the invention relates to compositions having ‘smart’ properties such as mucoadhesivity, oral bioavailability, and multifunctionality for systemic targeting, and, in preferred formulations which include curcumin in combination with one or more cancer therapeutics, which address resistance cancer therapy.

### BACKGROUND OF THE INVENTION

During the last two decades numerous drug delivery systems have been developed for hydrophobic and poorly water soluble medicines. These systems are focused on overcoming the poor availability of the drug and the subsequent ineffective therapy inherent to these types of molecules.

To solve the above mentioned problem associated with the solubilization of poorly water-soluble medicines, U.S. Pat. Nos. 5,645,856 and 6,096,338 disclose methods for preparing carriers for hydrophobic drugs, and pharmaceutical compositions based thereon, in which the carrier is comprised of biocompatible oil and a pharmaceutically acceptable surfactant component for dispersing the oil in vivo upon administration of the carrier. The amphiphilic surfactant component utilized does not substantially inhibit the in vivo lipolysis of the oil. These types of formulations can be utilized as a carrier system for many hydrophobic drugs resulting sometimes in enhanced bioavailability as compared with existing formulations of such drugs. However, these formulations are not stable in vivo and there is the possibility of drug leakage from the emulsion leading to unnecessary side effects in the body. Moreover, the surfactants used may disrupt the biological membranes causing cytotoxicity. In addition, targeting of a drug using such emulsion systems is not possible.

Other drug carriers have been used such as amphiphilic block copolymers which faun polymeric micelles or supramolecular assemblies wherein the hydrophobic part forms the core and the hydrophilic part the shell. The U.S. Pat. 5,510,103 describes block copolymers having the hydrophilic and hydrophobic segments forming micelles and entrapping the hydrophobic drugs by physical methods. The hydrophilic segment is preferably poly(ethylene oxide) and the hydrophobic segment is preferably poly(epsilon-benzyl-L-aspartate), while the preferred drug is Adriamycin.

Recently, polymeric micelles have been widely used as drug delivery carriers for parenteral administration. Micellar drug delivery carriers have several advantages including biocompatibility, solubilization of hydrophobic drugs in the core, manometric size ranges which facilitate extravasation of the drug carrier at the site of inflammation, site-specific delivery, etc. For example, U.S. Pat. No. 5,955,509 describes the use of poly(vinyl-N-heterocycle)-b-poly(alkylene oxide) copolymers in micelles containing pharmaceutical formulations. These copolymers respond to pH changes in the environment and can be used to deliver therapeutic compounds at lower pH values. These polymeric micelles remain intact at physiological pH, while they will release their content when exposed to a lower pH environment such as in tumor tissue.

A number of amphiphilic copolymers, having non-ionic and/or charged hydrophobic and hydrophilic segments, that form micelles are reported in the literature. For example, U.S. Pat. No. 6,322,817 discloses the injectable formulation of cross-linked polymeric micelles constituted by acrylic monomers—N-isopropylacrylamide, N-vinylpyrrolidoue and PEGylated monoesters of maleic acid. These polymeric nanoparticles are reported to have dissolved paclitaxel and delivered the drag to the tumor tissue through parenteral administration. However, these particles are only reported to be suitable for delivery via the intravenous route. Moreover, the reported use of alkylcyanoacrylate as one of the components in the copolymeric micelles may render the formulations toxic and unsuitable for in vivo applications.

One patent, U.S. Pat. No. 6,555,139 has disclosed a process of microfluidization or wet-micronization of hydrophobic drugs in combination with dextrins such as β-cyclodextrin. The patent indicated that the process of microfluidization facilitates the reduction of mean particle size of slightly soluble but highly permeable drugs, and creates a smooth, latex-like micro-suspension. A blend of expandable polymer and insoluble, hydrophilic excipients granulated with the micro-suspension create a matrix that after compaction erodes uniformly over a 24-hour period. However, the problems associated with these microfluidization systems are that for every molecule of drug, one molecule of β-cyclodextrin is required leading to large amounts of this compound to be administered inside the body along with drug. Moreover, drug leakage from β-cyclodextrin as well as poor bioavailability of β-cyclodextrin—drug complex has the potential to cause side effects. Finally, the particle size of up to 500 nm diameter may be responsible for limited utility for drug delivery purposes.

Another patent, U.S. Pat. No. 6,579,519 has disclosed the formulation of non-PEGylated pH sensitive and temperature sensitive cross-linked polymeric micelles constituted of N-isopropylacrylamide, acrylic acid and N-vinylpyrrolidone. These particles have extremely limited applications and can be used only for the specific purpose of topical delivery on the ocular surface. This is because of the fact that the LCST (lower critical solution temperature) of the particles is below ambient body temperature, and the particles are aggregated to a hydrophobic mass in vivo. Therefore, these particles are not suitable for systemic circulation and targeting, including oral delivery. Other similar patents are U.S. Pat. No. 6,746,635 and U.S. Pat. No. 6,824,791.

Another U.S. Pat. No. 7,094,810 describes a formulation which is composed of a hydrophilic segment made of poly(ethylene oxide) and a hydrophobic segment composed of vinyl monomers containing at least one pendant carboxyl group. More particularly, the vinyl monomers included in the polymer are acrylic acid or methacrylic acid having pendant carboxyl groups and butyl(alkyl)acrylate where the butyl segment can be a linear or branched chain. Thus, the hydrophobic segment is a mixture of non-ionizable butyl(alkyl)acrylate and ionizable(alkyl)acrylic acid which controls the hydrophobicity of the polymer. The ionizable carboxylic group of the polymer extended towards the surface of the particle is reported to be responsible for pH sensitivity.

Though the majority of these polymers can be used for injectable or topical delivery of bioactive agents, what are presently lacking are multifunctional amphiphilic polymers capable of oral delivery applications, by means of their nanoparticulate size and mucoadhesivity. The surface reactive functional groups of such “smart” nanoparticles would be capable of optional modification through PEGylation, ligand attachment, or fluorophore tagging for the purposes of systemic targeting, thus being useful for concurrent biological applications in diagnostics, therapeutics, and in imaging. Herein, we describe such an orally bioavailable smart polymeric nanoparticle system.

Choi, Cancer Lett 2008 Jan. 18, 259(1):111-8 describes the use of curcumin to down-regulate the multidrug-resistance mdr1b gene by inhibiting the P13K/Akt/NF kappa B pathway. Curcumin is a constituent of turmeric which anti-inflammatory, anti-carcinogenic, and chemopreventive effects in animal tumor models. Expression of P-glyocoprotein (p-gp) encoded by the mdr gene is associated with multidrug resistance (MDR) to unrelated chemotherapeutic drugs in cancer cells. Choi presents investigative results tending to demonstrate curcumin down regulates P-gp expression in multidrug-resistant L1210/Adr cells, and hypothesizes that curcumin may contribute to the reversal of the MDR phenotype. Choi does not discuss effective delivery of curcumin to a subject for addressing MDR or delivery chemotherapeutic drugs to the subject. It would be advantageous to provide a treatment modality where multidrug resistance to chemotherapeutic agents are effectively addressed using curcumin.

### SUMMARY OF THE INVENTION

An embodiment of the invention includes to cross-linked polymeric nanoparticles that are preferably 50-100 nm or smaller in size (preferably less than 5% having a diameter in excess of 200 nm) which include curcumin on entrapped within the hydrophobic interior of the nano particles and one or more chemotherapeutic agents (e.g., anthracyclines (doxorubicin, daunorubicin), paclitaxel and other taxanes, cisplatin and other platinum compounds, topoisomerase inhibitors, etc.), preferably bound or conjugated to or otherwise associated with the nanoparticles (preferably on an exterior surface of the nanoparticles), where the nanoparticles comprise a polymeric substrate formed from monomers consisting of N-isopropylacrylamide (NIPAAM), acrylic acid (AA), and at least one vinyl monomer selected from the group consisting of vinyl acetate, 4-vinyl benzoic acid, methylmethacrylate, vinylmethacrylate, N-vinylpyrrolidone, N-vinyl piperidone, N-vinyl caprolacum, N-vinyl carbazole, and styrene, wherein said NIPAAM, said AA, and said vinyl monomer are present at molar ratios of 50-70:10-30:10-30 for NIPAAM:AA:vinyl monomer.

A further embodiment of the invention is to utilize the nanoparticles to treat a subject (human or animal) with the nanoparticles, or compositions including the nanoparticles distributed within (e.g., dispersed) a carrier fluid (e.g., water, oil, or other suitable fluid), where the curcumin functions to overcome the multidrug resistance to the cancer chemotherapeutics and treatment related systemic toxicity.

Still further, an embodiment of the invention is to provide a method of making the chemotherapeutic nanoparticles.

Yet another object of this invention is to provide a process for the preparation of nanoparticles incorporating the combinations of medicines, with the option of chemically conjugating polyethylene glycol (PEG) chains of varying chain length (50-8000 D) at the outer surface of the nanoparticles to reactive moieties on the surface of formed nanoparticles, where the PEG chains can help the particles to circulate in the blood for a relatively long time, following systemic administration.

According to the invention, medicinal compositions are prepared which comprise polymeric nanoparticles preferably of a size on average of less than 100 nm diameter entrapping curcumin in combination with one or more cancer chemotherapeutic agents. These amphiphilic nanoparticles can be made of cross-linked polymers which are mainly composed of the following three constituents added as monomers at specific molar ratios: (1) N-isopropylacrylamide (NIPAAM), plus (2) either a water-soluble vinyl compound like vinyl acetate (VA) or vinyl pyrrolidone (VP), so as to make the particle shell more hydrophilic, or a water-insoluble vinyl derivative such as styrene (ST) or methylmethacrylate (MMA), so as to make the particle core more hydrophobic, plus (3) acrylic acid (AA), which provides surface reactive functional groups. The surface of the nanoparticles can be optionally functionalized using the reactive functional groups provided by AA, including by PEGylation for long circulation in blood, or by addition of other surface reactive groups which can be used for targeting to tissues in viva for therapeutic, diagnostic, and imaging applications.

Resistance to cancer chemotherapy is a major cause for treatment failure and disease progression in cancer. One of the most important reasons for treatment resistance is the development of multidrug resistance (MDR) phenotype, which arises as a result of upregulation of various drug efflux transporter proteins. There are three major drug efflux transport proteins in human cancers: MDR1/ABCB1 (a.k.a P-glycoprotein), MRP-1/ABCC1 and ABCG2/BCRP. Upregulation of various MDR proteins is observed in many human cancers, particularly in advanced disease, which results in efflux of commonly used chemotherapeutic agents administered in these cancers, such as the anthracyclines (doxorubicin, daunorubicin), paclitaxel and other taxanes, cisplatin and other platinum compounds, topoisomerase inhibitors, etc. While MDR can be overcome to some extent by using higher dosages of chemotherapeutics, this in turn, can lead to systemic side effects in other organs, such as cardiotoxicity, nephrotoxicity, gastrointestinal toxicity, and bone marrow suppression, amongst others. A formulation that can overcome the MDR phenotype in cancers, while at the same reducing systemic adverse effects (i.e. killing two birds with one stone), would be of considerable value in clinical oncology. An embodiment of this invention presents a formulation of a composite polymeric nanoparticle that comprises curcumin in its hydrophobic core, and doxorubicin conjugated to the hydrophilic surface (NanoDoxCurc). Curcumin, derived from the Indian spice turmeric, is a potent inhibitor of all three MDR proteins, and allows the doxorubicin to accumulate within its site of action (the nucleus) in cancer cells without being effluxed. In multiple cancer models with high MDR protein expression and specifically selected for doxorubicin resistance (human prostate cancer, human multiple myeloma, human ovarian cancer, and murine leukemia), NanoDoxCurc is able to overcome the MDR phenotype, and either induce xenograft regression or significantly enhance survival compare to doxorubicin formulation alone. Notably, the effects of NanoDoxCurc are observed irrespective of the MDR protein expressed, suggesting that curcumin is a potent “pan-inhibitor” of all three MDR proteins. Importantly, in addition to its effects on cancer cells, we also observe that NanoDoxCurc is able to significantly attenuate the systemic adverse effects of doxorubicin on other organs systems, particularly the heart and bone marrow. This is highly clinically significant because one of the most reasons for dose limiting toxicity with doxorubicin is its adverse effect on the myocardium, with long term cardiomyopathy developing in patients who receive greater than a certain cumulative dose of the drug. In animal studies, using equivalent doses of free doxorubicin, pegylated liposomal doxorubicin (Doxil) and NanoDoxCurc we observe unequivocal echocardiographic evidence of cardiac toxicity with both doxorubicin and Doxil, while NanoDoxCurc demonstrates no evidence of cardiac side effects. Similarly, we observe clear cut evidence of hematological toxicity with doxorubicin and Doxil, while NanoDoxCurc shows no effects on the bone marrow at equivalent doses. Investigations have also confirmed that curcumin attenuates reactive oxygen species (ROS) induced cardiomyocyte damage, which are a byproduct in the heart of doxorubicin administration. Thus, in a preferred embodiment, the invention is a composite nanoparticle that serves a dual purpose of (a) overcoming MDR phenotype in cancer cells induced by multiple MDR proteins, while at the same time (b) reducing systemic adverse effects of the chemotherapeutic (doxorubicin in the example) in non-cancerous tissues. In addition to improving the efficacy of chemotherapeutics in advanced cancer, this composite nanoparticle should allow an increase in cumulative dose of chemotherapeutics that can be administered without an amplification of adverse effects.

### BRIEF DESCRIPTION OF THE FIGURES

FIG. 1 illustrates a polymeric nanoparticle with the hydrophobic core (10) composed of hydrophobic parts of the polymers entrapping the medicine (11), the hydrophilic parts forming a hydrophilic shell (12) which are present towards the aqueous medium. The nanoparticles are less than 100 nm size, and may include one or more molecules of medicaments or other bioactive agents.

FIG. 2 illustrates three examples of poorly water soluble drugs whose solubilization has been enabled by entrapment in polymeric nanoparticles embodied in this invention. Free paclitaxel (taxol) (A), free rapamycin (C), and free rifampicin (E) are essentially insoluble in water, as evidenced by turbidity of solution and visible floating particles of each drug. In contrast, equivalent amounts of nanoparticle-encapsulated paclitaxel (B), nanoparticle-encapsulated rapamycin (D), and nanoparticle-encapsulated rifampicin (F) form transparent solutions in water.

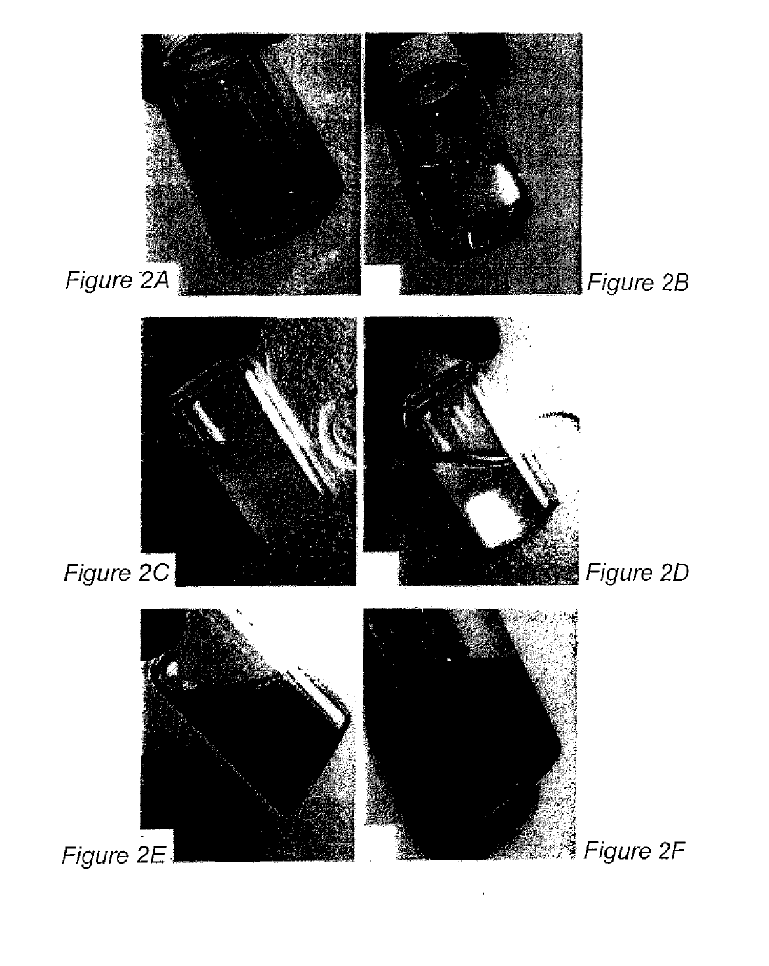

FIG. 3 shows lower critical solution temperature (LCST) as a function of the weight percent ratio of the constituents, and in particular the molar ratio of NIPAAM in the nanoparticles. In the illustrated example, three different compositions of nanoparticles are represented, each with a different molar ratio of NIPAAM (NP), vinyl pyrrolidone (VP)and acrylic acid (AA) comprising the polymeric nanoparticles. Average size of nanoparticles (nm) is measured by dynamic light scattering and other methods. Compositions with a NIPAAM molar ratio of 90% have a LCST below that of body temperature, while compositions with a NIPAAM molar ratio of 60% has a LCST above that of body temperature.

FIG. 4a is a Transmission Electron Microscopy (TEM) photomicrograph of NIPAAM/VP/AA polymeric nanoparticles (molar ratios of 60:20:20), which have an average diameter of 50 nm or less (100 nm scale is illustrated at bottom right). FIG. 4b is a TEM photomicrograph of NIPAAM/MMA/AA polymeric nanoparticles (molar ratios of 60:20:20), which have an average diameter of 50 nm or less (500 nm scale is illustrated at bottom right). Minimal polydispersity is observed.

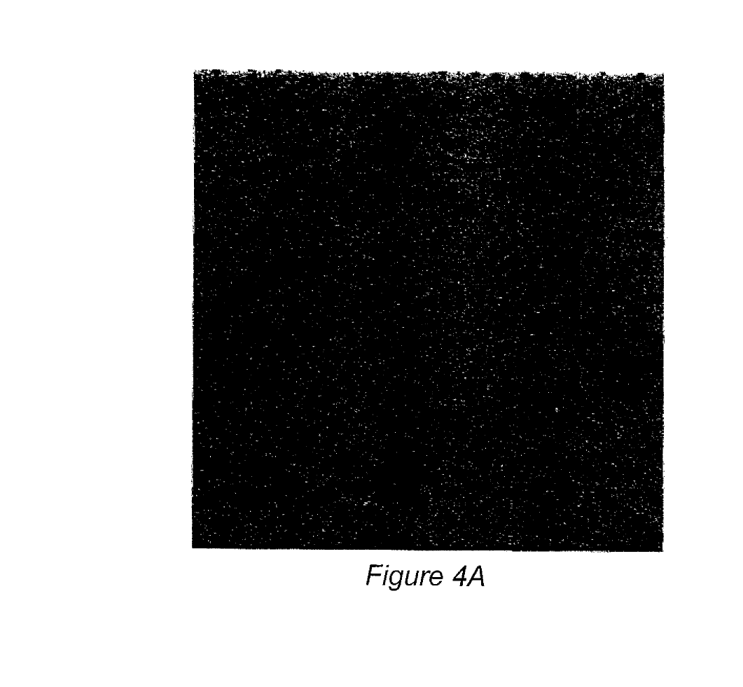

FIGS. 5a-c illustrate lack of demonstrable in vivo toxicity from orally delivered empty (“void”) polymeric nanoparticles. Two types of orally delivered void nanoparticles were utilized: NIPAAM/VP/AA in molar ratios of 60:20:20 (designated NVA622) and NIPAAM/MMA/AA in molar ratios of 60:20:20 (designated NMA622). Groups of four CD1 wild type mice each (two males, two females) were administered 500 mg/kg of void NVA622 or void NMA622 nanoparticles in 500 μL of of water, five consecutive days a week, for two weeks. During and at the culmination of void nanoparticle administration, no weight loss, behavioral abnormalities or other abnormal features were seen. No gross (macroscopic) toxicities were observed in the mice receiving either the void NVA622 or the void NMA622 nanoparticles.

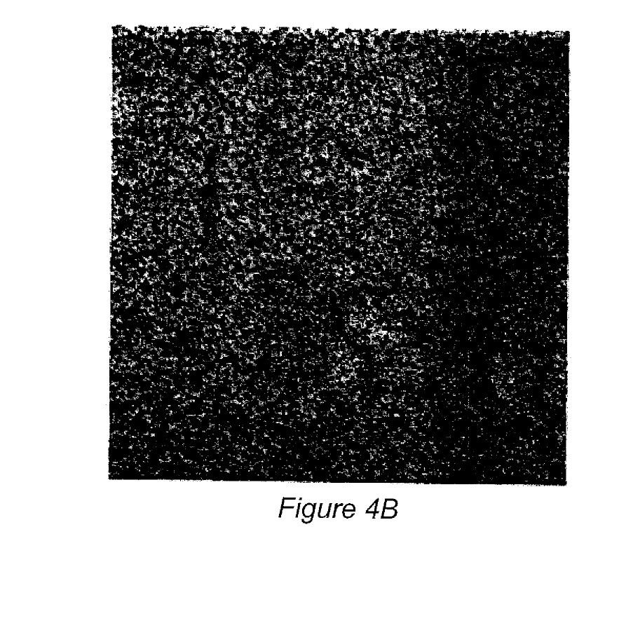

FIG. 6 illustrates in vitro cell viability (3-(4,5-Dimethylthiazol-2-yl)-2,5-diphenyltetrazolium bromide, or MTT) assays performed with polymeric nanoparticle encapsulated paclitaxel (nanopaclitaxel), and comparison with free paclitaxel. In the illustrated example, NIPAAM/VP/AA polymeric nanoparticles in molar ratio of 60:20:20 were used for paclitaxel encapsulation. Three human pancreatic cancer cell lines (XPA-1, BxPC3 and PANC-1) were incubated with increasing concentrations (1, 10, 20, 50, and 100 nm) of either free paclitaxel (black bar) or equivalent amount of nanopaclitaxel (grey bar) for 48 hours. Also included as control in each condition were void polymeric nanoparticle equal to the amount required for encapsulating said dose of nanopaclitaxel (white bar) and solvent (dimethylsulfoxide [DMSO], blue bar) equal to the amount required for dissolving said dose of free paclitaxel. Nanopaclitaxel (grey bar) demonstrates comparable cytotoxicity in all three cell lines in vitro, compared to free paclitaxel (black bar). Thus, nano-encapsulation of the drug is not associated with loss of drug activity. In contrast, and as expected, treatment with the void polymer only does not demonstrate any significant effect of cytotoxicity compared to baseline control growth of the cells (0 nm condition). All assays were performed in triplicate and error bars represent standard deviations.

FIG. 7 illustrates in vitro cell viability (MTT) assays performed to demonstrate the synergistic effects of polymeric nanoparticle encapsulated paclitaxel (nanopaclitaxel) and polymeric nanoparticle encapsulated curcumin (nanocurcumin). Three human pancreatic cancer cell lines (XPA-1, BxPC3 and PANC-1) were incubated with increasing concentrations (1, 2, 4, 6, 8 and 10 nm) of either free paclitaxel (black bar) or equivalent amount of nanopaclitaxel (white bar) for 48 hours. In order to test therapeutic synergy with curcumin, the cells were also incubated with either free curcumin (15 μM) plus free paclitaxel (grey bar), or with equivalent amount of nanocurcumin (15 μM) plus nanopaclitaxel (blue bar). As illustrated, the combination of nanopaclitaxel and nanocurcumin demonstrates increased cytotoxicity than either free paclitaxel or nanopaclitaxel alone at any given dose of paclitaxel. Of note, and especially at the lower dosages used in two of the cell lines (XPA-1 and Panc-1), the combination of nanopaclitaxel and nanocurcumin also appears to have better efficacy than the combination of free paclitaxel and free curcumin, likely due to increased intracellular uptake of the nano-encapsulated compounds. At higher dosages, the combination therapy with either free or nano-encapsulated drugs appears to have comparable effects.

FIG. 8 illustrates the bactericidal effects of nanoparticle encapsulated rifampicin and free rifampicin against Mycobacterium tuberculosis (MTB). In this experiment, MTB was cultured for two weeks in absence of any treatment, nano-encapsulated rifampicin, free rifampicin, and void nanoparticles. There is robust MTB growth in the no treatment and in the void nanoparticle tubes, the latter consistent with lack of toxicity from the polymer per se. In contrast, MTB growth is completely inhibited in the nano-encapsulated rifampicin and free rifampicin tubes.

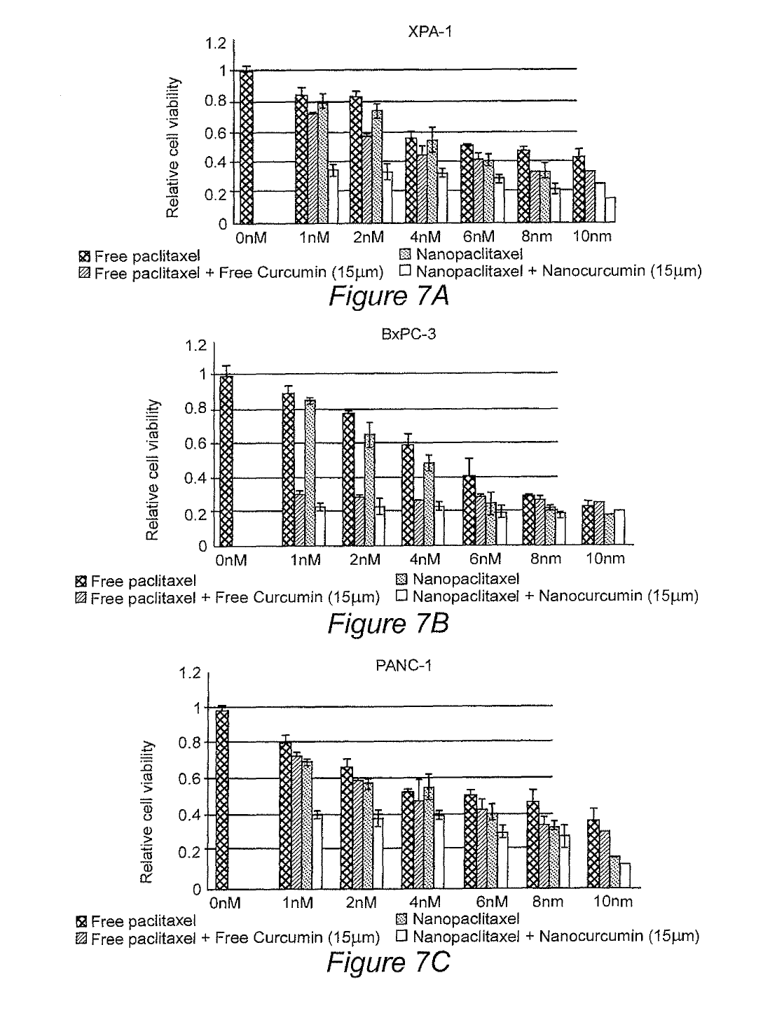

FIG. 9 illustrates in vitro cell viability (MTT assay) performed using the water-soluble drug gemcitabine conjugated to the acrylic acid (AA) surface reactive functional group of polymeric nanoparticle. Unlike the poorly water drugs that are encapsulated within the nanoparticle, water soluble drugs like gemcitabine can be conjugated to the nanoparticle surface, rendering this compound amenable to oral delivery. Human pancreatic cancer cell line BxPC3 is incubated with increasing dosages of either free gemcitabine (black bar), nano-gemcitabine (white bar), void polymer (grey bar), or PBS solvent (patterned bar). UT=untreated. At 96 hours, free gemcitabine and nano-gemcitabine demonstrated comparable activity. All assays were performed in triplicate and means and standard deviations are plotted.

FIG. 10 illustrates blood levels of rapamycin following oral delivery of polymeric nanoparticles. Rapamycin was encapsulated in nanoparticles comprised of increasing order of acrylic acid (AA) percentage in the co-polymeric composition. The nanoparticles were either administered as is, or after surface PEGylation. Compared are: Control A (rapamycin suspended in water); rapamycin nanoparticle comprised of NIPAAM:VP:AA in a ratio of 60:30:10 (designated as NVA631); rapamycin nanoparticle comprised of NIPAAM:VP:AA in a ratio of 60:20:20 (designated as NVA622); rapamycin nanoparticle comprised of NIPAAM:VP:AA in a ratio of 60:10:30 (designated as NVA613); and rapamycin nanoparticle comprised of NIPAAM:MMA:AA in a ratio of 60:20:20 (designated as NMA622). The corresponding PEGylated nanoparticles (PEG-NVA-631, PEG-NVA-622, PEG-NVA-613, and PEG-NMA-622) encapsulating rapamycin are designated as shaded bars. Rapamycin was administered either as free drug dispersed in water (15 mg/kg) or as equivalent dosage of nano-encapsulated rapamycin in the respective polymeric nanoparticle formulation. Six wild type C57/B6 mice were included in each arm of this study. Blood levels are measured by HPLC from samples obtained at 2 hours post oral delivery. Two types of nanoparticles, each containing 20% molar ratio of AA (NVA622 and NMA622) demonstrate highest blood levels of rapamycin following oral delivery.

FIG. 11 illustrates pharmacokinetic (PK) data of orally delivered nano-encapsulated rapamycin in mice, over a 24 hour period. Two polymeric nanoparticle formulations with highest blood levels at 2 hours (FIG. 10) were selected for this study: NVA622 and NMA622, containing NIPAAM/VP/AA and NIPAAM/MMA/AA in 60:20:20 molar ratios, respectively. Six wild type C57/B6 mice were included in each arm of the study. Single dose of nano-encapsulated rapamycin (equivalent to 15 mg/kg of drug) was administered at time zero, and blood obtained from the facial vein by venupuncture, at 30 minutes, 2, 4, 8, and 24 hours post oral administration. Rapamycin levels were measured by HPLC on mouse plasma. The means and standard deviations (error bars) are plotted for each time point for each of the nanoparticle formulations. NMA622 nanoparticles have a higher area-under-the-curve (AUC) compared with NVA622 nanoparticles (Mean AUC 26,949 versus 11,684, respectively).

FIG. 12 illustrates levels of rapamycin in central and peripheral venous circulation at 2 hours post-administration of nanoparticle encapsulated rapamycin via oral route. NVA622 particles encapsulating rapamycin were administered via oral route in three mice (dose of 15 mg/kg) and rapamycin levels measured in central venous and peripheral venous (facial vein) circulation at 2 hours. The levels are identical in all three independent measurements between the two sites, consistent with equitable systemic distribution of the orally delivered nanoparticle-encapsulated rapamycin within the blood circulation.

FIG. 13 is a schematic diagram showing the synthesis of NanoCurc, NanoDox, and NanoCurcDox (NCD) (an exemplary nanoparticle according to the present invention).

FIG. 14a-c are bar graphs showing cytotoxic efficacy of NanoDox, NanoCurc, and NanoDoxCurc towards various DOX resistant clones. Cell viability (MTT) assays were performed in three different DOX resistant cell lines: NCI/ADR (FIG. 14a), PC-3A (FIG. 14b), and RPM18226/Dox (FIG. 14c). NDC significantly inhibited the growth of all three DOX resistant cancer cell lines relative to control, ND and NC (*p<0.0001).

FIGS. 15a-c. NDC overcomes DOX resistance in vivo.(a) side by side graphs and images which show NDC significantly inhibits the growth of subcutaneous DOX resistant cancer xenografts. Subcutaneous xenografts were established using the PC-3A human DOX resistant prostate cancer cell line or RPMI8226/Dox human myeloma cell line, and mice were randomized to four arms, including (1) vehicle control (ii) ND, (iii) NDC, and (iv) NC. Representative excised xenograft tumors from each of the four arms are illustrated. NDC significantly blocked tumor growth compared to either ND or NC. Graph shows mean tumor volume+/−S.E.M. n=5, *P<0.05 compared to NC. (b) bar graphs which show no reduction in body weight was observed in any treated arm over the course of treatment in the PC-3A and RPMI8226/Dox cell lines. (c) P388/ADR ascites fluids were injected intraperitoneally in BDF1 syngenic mice and mice were randomized to three arms: (i) vehicle control, (ii) ND, and (iii) NDC. Graph shows greater than 50% increase in survival was observed in NDC treated mice compared to ND or vehicle treated mice. n=8, *P<0.005.

FIG. 16a-d. Doxorubicin formulation NDC has no observable cardiotoxicity. C57BL6/J mice received an equivalent weekly cumulative dose of free doxorubicin, commercially available liposomal formulation of doxorubicin (Doxil), NanoDox, NanoDoxCurc, or vehicle only for 4 weeks. One week after the last dose, various cardiac parameters were examined by echocardiogram followed by collection of blood by cardiac puncture. Heart sections were examined by histology and several molecular markers of cardiac cells were examined by western blot. (a) Representative echocardiogram graph and (b) cardiac parameters were plotted graphically. (c) Body weight and heart weight of mice receiving each treatment. (d) Hemoglobin and leukocyte counts from blood samples from each treatment arm.

### DETAILED DESCRIPTION OF THE INVENTION

Medicinal compositions of poorly water-soluble medicines, alone or in combination with two or more medicines, entrapped into polymeric nanoparticles are described herein. Medicinal composition of water-soluble medicines such as gemcitabine conjugated to a surface of polymeric nanoparticles are also described herein. After formation, the nanoparticles are approximately spherical and preferably have a size that averages 50-100 nm or less in diameter. The nanoparticles may be described as nanometer sized particles of micellar aggregates of amphiphilic and cross-linked polymers.

In an embodiment of the present invention, nanoparticles of polymeric micelles are prepared by:
(i) dissolving NIPAAM and AA in water to form micelles,
(ii) adding at least one compound of vinyl derivative, which may be either water-soluble or insoluble in water, but both are soluble in the said micelles and which can be polymerized through free radical polymerization,
(iii) adding appropriate amount of activator and initiator, which are, for example, tetramethylethylene diamine (TMED) and ferrous ammonium sulphate. As activators and ammonium perdisulphate as activator.
(iv) adding a cross-linking agent to the said micellar solution, which is preferably N,N′ methylene bis acrylamide
(v) polymerizing the monomers into copolymer in presence of an inert gas such as nitrogen at 30 C to 40 C temperature for 24 hours for nearly completion of the reaction,
(vi) purifying the nanoparticles of the co-polymeric micelles by dialysis for three hours to remove toxic monomers and other unreacted materials,
(vii) optional surface modification of the nanoparticles by chemically conjugating PEG amine of variable chain length (50-8000 D) or other conjugated moieties to reactive functional groups on the nanoparticle surface,
(viii) addition of one or more bioactive agents for entrapment within the formed polymeric nanoparticles in aqueous solution, or lyophilizing the empty polymeric nanoparticles to dry powder for future use,
(ix) reconstituting the dry powder of empty polymeric nanoparticles in an aqueous solution, and addition of one or more bioactive agents for entrapment within the said polymeric nanoparticles,
(x) lyophilizing the drug-loaded polymeric nanoparticles to dry powder, and
(xi) reconstituting the drug loaded polymeric nanoparticles in aqueous solution for oral, injectable, or topical delivery.

Besides NIPAAM and AA, the vinyl monomers are selected from water soluble vinyl compounds such as vinyl acetate, 4-vinyl benzoic acid, N-vinylpyrrolidone (VP), and N-vinyl piperidone, while water insoluble amphiphilic vinyl compounds include methylmethacrylate (MMA), vinylmethacrylate, N-vinyl caprolactam, N-vinyl carbazole, and styrene.

In one embodiment, the nanoparticles are formed by polymerization of the monomers in the reaction mixture. The compositions are in the following molar ratios: NIPAAM, about 50% to about 90%, and preferably 60% for specific delivery routes such as oral or parenteral; a vinyl monomer like the water-soluble VP or water-insoluble MMA, about 10% to about 30%; and AA, about 10% to about 30%. The monomers are dissolved in water and ammonium perdisulphate TEMED and ferrous ammonium sulphate are added to it. N,N′ methylene bis acrylamide is also added to cross-linked the polymer. The mixture is permitted to polymerize, preferably in the presence of an inert gas (e.g., nitrogen, argon, etc.), at a temperature preferably ranging from 20° C. to 80° C., or more preferably from 30° C. to 40° C., until polymerization is complete. Completion of polymerization may be determined by depletion of monomers from the reaction mixture by HPLC or ¹H NMR of vinyl protons. The solution may be purified by dialysis, for example for 2-4 hours, to remove any toxic monomers or other unreacted species. In Example 1, NIPAAM, VP, and AA were used to prepare copolymers with the molar ratios of 60:30:10, 60:20:20, and 60:10:30, in order to potentially modulate the mucoadhesivity of orally delivered nanoparticles in the GI tract by varying the proportion of AA in the polymer. In Example 2, similar co-polymeric nanoparticles were prepared in which VP has been replaced by MMA, and in the specific example the molar ratios used was 60:20:20 for NIPAAM, MMA and AA, respectively. As will be discussed below, the proportion of monomers utilized also affects stability of the nanoparticles at body temperature.

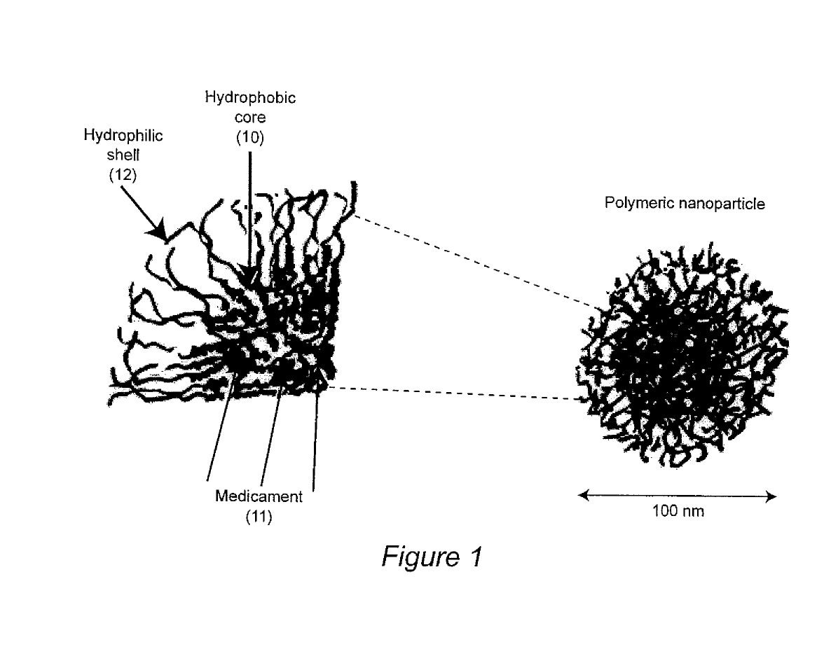

One embodiment of the invention is illustrated in FIG. 1, which shows that the nanoparticles have a hydrophobic core (labeled 10) composed of hydrophobic parts of the polymers entrapping the medicine (labeled 11), whereas the hydrophilic parts forming a hydrophilic shell (labeled 12) are present towards the aqueous medium. As also shown in FIG. 1, the polymeric nanoparticles are preferably less than100 nm in size, and may include one or more molecules of medicaments or other bioactive agents.

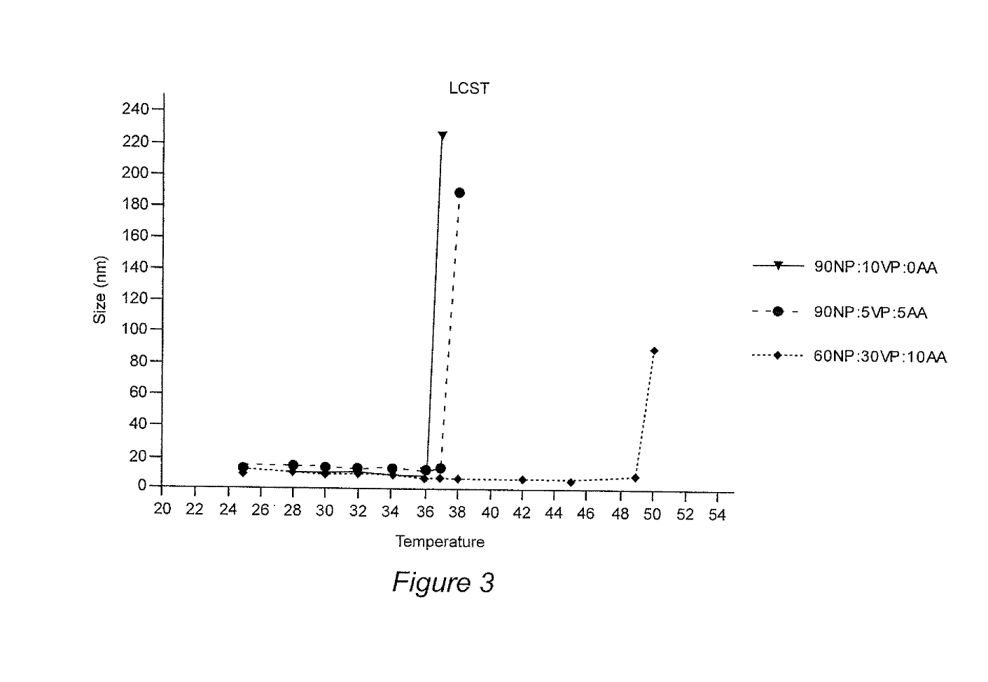

Due to the presence of NIPAAM in the copolymeric formulation, the nanoparticle shell is converted from a hydrophilic to a hydrophobic entity at the lower critical solution temperature (LCST), which can be modulated by changing the amount of NIPAAM in the proportion of monomers used, as seen in FIG. 3. To render these nanoparticles suitable for systemic circulation, the nanoparticles should have a LCST above human body temperature (˜37° C.). In order to obtain a high LCST of the nanoparticles, i.e., in the 45-50° C. range, enabling systemic medicine delivery and stability of the nanoparticles at body temperature, it is required that the NIPAAM component be used in an optimum molar ratio of 50-70%, with the two remaining monomers comprising the remaining 100%. As noted above, additional monomers or functional moieties may also be included, and these do not impact the LCST.

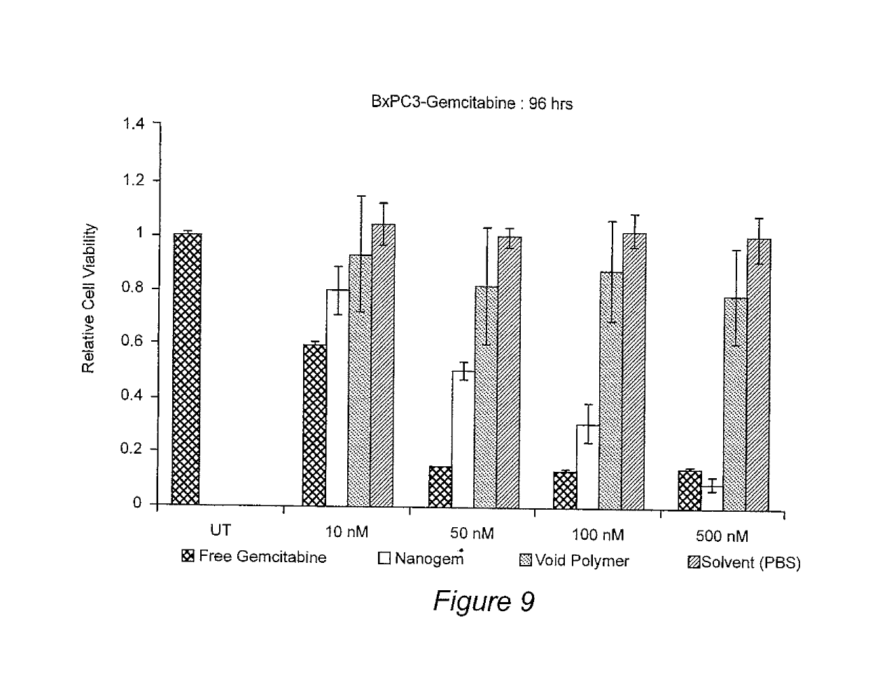

The nanoparticles described herein can be used as is for drug delivery, or optionally, the surface of nanoparticles may be modified by functionalizing reactive surface groups (COO—) provided by AA for attachment of PEG amine chains of variable length (50-8000 D), or for the chemical conjugation of targeting moieties like ligands, antibodies, radionuclides, fluorophores, and contrast agents, or for the addition of taste masking agents like aspartame. The addition of PEG amine chains does not impede the observed oral bioavailability of the drug encapsulated nanoparticles, as seen in FIG. 10. Herein, four independent nanoparticle formulations encapsulating rapamycin (NVA631, NVA622, NVA613, and NMA622) were administered to mice via oral route, and the drug levels at two hours in the systemic circulation compared with that of rapamycin encapsulated in corresponding PEGylated nanoparticles (PEG-NVA613, PEG-NVA622, PEG-NVA613, and PEG-NMA622). As seen, the blood levels of rapamycin following oral delivery of non-PEGylated and PEGylated nanoparticles are comparable. Those skilled in the art will be aware that PEGylation renders nanoparticle long circulating, by evading the innate reticuloendothelial system (RES), and the engineering of “RES evading” nanoparticles embodied in this invention does not impede their oral bioavailability.

The polymeric nanoparticles disclosed herein are preferably loaded with medicines or other bioactive agents to the maximum extent possible. The medicines or bioactive agents can be organic compounds that are poorly soluble or insoluble in water but readily soluble in organic solvents. The medicine or bioactive agent is added to the polymeric solution either in the form of dry powder or as a solution in chloroform, ethanol or ether depending on the solubility of the drug in that solvent to form an optically clear solution. Examples of such medicines include, but are not limited to, antineoplastic agents such as Paclitaxel, Docetaxel, Rapamycin, Doxorubicin, Daunorubicin, Idarubicin, Epirubicin, Capecitabine, Mitomycin C, Amsacrine, Busulfan, Tretinoin, Etoposide, Chlorambucil, Chlormethine, Melphalan, and Benzylphenylurea (BPU) compounds; phytochemicals and other natural compounds such as curcumin, curcuminoids, and other flavinoids; steroidal compounds such as natural and synthetic steroids, and steroid derivatives like cyclopamine; antiviral agents such as Aciclovir, Indinavir, Lamivudine, Stavudine, Nevirapine, Ritonavir, Ganciclovir, Saquinavir, Lopinavir, Nelfinavir; antifungal agents such as Itraconazole, Ketoconazole, Miconazole, Oxiconazole, Sertaconazole, Amphotericin B, and Griseofulvin; antibacterial agents such as quinolones including Ciprofloxacin, Ofloxacin, Moxifloxacin, Methoxyfloxacin, Pefloxacin, Norfloxacin, Sparfloxacin, Temafloxacin, Levofloxacin, Lomefloxacin, Cinoxacin; antibacterial agents such as penicillins including Cloxacillin, Benzylpenicillin, Phenylmethoxypenicillin; antibacterial agents such as aminoglycosides including Erythromycin and other macrolides; antitubercular agents such as rifampicin and rifapentin; and anti-inflammatory agents such as Ibuprofen, Indomethacin, Ketoprofen, Naproxen, Oxaprozin, Piroxicam, Sulindac. Preferably, the medicine(s) loaded in the compositions range from 1% to 20% (w/w) of the polymer; however, in some applications the loading may be considerably higher.

Generally, one or more bioactive agents, such as medicines which are poorly soluble in aqueous media but also including other agents that produce a biological effect, are dissolved in a suitable solvent, such as ethanol or chloroform, and added to a nanoparticle solution. This addition step can be performed before or after nanoparticle formation. Combining the medicines or bioactive agents with the nanoparticle solution results in the entrapment of the medicines or bioactive agents within the hydrophobic core (interior) of the nanoparticles. The nanoparticles containing the entrapped medicines or bioactive agents may, if desired, be lyophilized or otherwise rendered into powder form for subsequent reconstitution in a suitable fluid vehicle for human or mammalian administration. In the subsequently discussed Example 5, incorporating FIGS. 10, 11, and 12, the in vivo oral bioavailability of rapamycin encapsulated in polymeric nanoparticles is demonstrated.

In another embodiment of this invention, a medication, which is water soluble but otherwise has low bioavailability through the oral route, can be attached to the surface of the nanoparticles by covalent conjugation between the reactive carboxylic groups in the nanoparticle and complementary functional groups, such as amine or thiol groups, on the medication. Conjugation to the nanoparticles allows such medications to become orally bioavailable. Examples of such compounds include, but are not limited to, anti-neoplastic agents like gemcitabine.

The nanoparticles containing at least one medicine or a combination of medicines and bioactive agents prepared by the above described process (e.g., nanoparticles with entrapped medicines or medicines conjugated to a surface, or even combinations of both) may be used for the treatment of pathological conditions arising out of various diseases including but not limited to cancer, inflammation, infection and neurodegeneration.

The NanoDoxCurc embodiment of the invention solves two pervasive problems in clinical oncology. First, cancers overexpress a variety of MDR proteins that efflux chemotherapeutics out of the site of intracellular action, and lead to chemoresistance, resulting in treatment failure. Second, many of these chemotherapeutic agents cause incidental toxicity, such as cardiac and hematological side effects, leading to a threshold cumulative dose in humans that cannot be exceeded without the advent of side effects. Here, an advantage of this embodiment of the invention is that it “kills two birds with one stone” by the use of a novel composite polymer nanoparticle that bypasses chemoresistance and also overcomes many of the systemic side effects associated with chemotherapy, especially those on the heart and bone marrow.

The novel features of NanoDoxCurc (as well as related embodiments which include curcumin in combination with other chemotherapeutic agents) are (a) a polymer nanoparticle comprised of three monomers that are used in many FDA-approved products; (b) the ability to deliver two agents simultaneously; (b) one of the agents is a hydrophobic drug (curcumin) and encapsulated in the hydrophobic core of the nanoparticle; (c) the second dug (doxorubicin in the exemplary embodiment (but which could be other chemotherapeutic agents, as a well as a plurality of different chemotherapeutic agents) is conjugated to the surface of the nanoparticle (in some embodiments, a chemotherapeutic agent may be present in the core with curcumin); (d) curcumin delivered within the composite nanoparticle inactivates multiple MDR proteins (including MDR-1/PgP, MRP-1 and ABCG2/BCRP1) in cancer cells, thus allowing the concomitantly delivered doxorubicin to reach its site of action; (e) curcumin ameliorates the reactive oxygen species (ROS) mediated adverse effects of doxorubicin in non-cancerous tissues, mainly through enhancing cellular anti-oxidant levels and reducing oxidative stress. Importantly, preclinical studies show that dose-for-dose, NanoDoxCurc performs better in terms of reduced adverse effects compared to not only free doxorubicin, but also Doxi® 1 (pegylated liposomal doxorubicin) which is marketed specifically for the purpose of reducing the adverse effects of the drug. Thus, the new formulation outperforms Doxil® in terms of its safety profile in the preclinical small animal setting.

Doxorubicin is widely used as a cancer chemotherapeutic in many cancer regimens, both for solid malignancies as well as hematological cancers. It is especially used in many pediatric malignancy regimens like leukemias, where long term effects of cardiac toxicity can be devastating. The NanoDoxCurc formulation has a dual advantage, not only overcoming chemoresistance, but also reducing systemic adverse effects of the delivered chemotherapeutic. The scope of this invention extends to a substantial market that is currently occupied by doxorubicin or Doxil®, or where these two drugs failed to win regulatory approval due to toxicity issues.

The invention will now be described with reference to the following non-limiting examples:

### EXAMPLES

### Example1

### Synthesis of Cross-Linked Copolymeric Micelles of NIPAAM, VP (A Water-Soluble Vinyl Derivative), and AA

A co-polymer of NIPAAM with VP and AA was synthesized through free radical polymerization. Water-soluble monomers, NIPAAM, VP and AA were dissolved in water in 60:30:10 molar ratios for NVA631, 60:20:20 for NVA622, and 60:10:30 for NVA613. The polymerization was initiated using ammonium persulphate (APS) as initiator in N₂ atmosphere. Ferrous Ammonium Sulphate (FAS) was added to activate the polymerization reaction and also to ensure complete polymerization of the monomers to obtain a good yield. Using NVA631 as a prototypal example, in a typical experimental protocol, 62.8 mg of re-crystallized NIPAAM, 30.5 μl of freshly distilled VP and 6.61 μl of AA (freshly distilled) in 10 ml of water were used. To cross-link the polymer chain, 30 μl of MBA (0.049 g/ml) was added in the aqueous solution of monomers. Dissolved oxygen was removed by passing nitrogen gas for 30 minutes. 20 μl of FAS (0.5% w/v), 30 μl of APS and 20 μl of TEMED were then added to initiate the polymerization reaction. The polymerization was carried out at 30° C. for 24 hours in a nitrogen atmosphere. After the polymerization was complete, the total aqueous solution of polymer was dialyzed overnight using a spectrapore membrane dialysis bag (12 kD cut off). The dialyzed solution was then lyophilized immediately to obtain a dry powder for subsequent use, which is easily re-dispersible in aqueous buffer. The yield of the polymeric nanoparticle was more than 90%. When VP is replaced by other water-soluble vinyl derivatives like vinyl alcohol (VA), the method of preparation remains the same, and the co-polymer does not change in its properties.

### Example2

### Synthesis of Cross-Linked Copolymeric Micelles of NIPAAM, MMA (Water-Insoluble Vinyl Derivative), and AA

A co-polymer of NIPAAM with MMA and AA was synthesized through free radical polymerization. Water-soluble monomers—NIPAAM and AA—were dissolved in water, and water-insoluble MMA was dissolved in the micellar solution of NIPAAM and AA, in 60:30:10 molar ratios for NMA631, 60:20:20 for NMA622, and 60:10:30 for NMA613. The polymerization was initiated using ammonium persulphate (APS) as initiator in N₂ atmosphere. Ferrous Ammonium Sulphate (FAS) was added to activate the polymerization reaction and also to ensure complete polymerization of the monomers to obtain a good yield. Using NMA622 as a prototypal example, in a typical experimental protocol for preparing NMA622, 66.6 mg of re-crystallized NIPAAM, 19.4 μl of freshly distilled MMA and 14 μl of AA (freshly distilled) in 10 ml of water were used. To cross-link the polymer chain, 30 μl of MBA (0.049 g/ml) was added in the aqueous solution of monomers. Dissolved oxygen was removed by passing nitrogen gas for 30 minutes. 20 μl of FAS (0.5% w/v), 30 μl of APS and 20 μl of TEMED were then added to initiate the polymerization reaction. The polymerization was carried out at 30° C. for 24 hours in a nitrogen atmosphere. After the polymerization was complete, the total aqueous solution of polymer was dialyzed overnight using a spectrapore membrane dialysis bag (12 kD cut off). The dialyzed solution was then lyophilized immediately to obtain a dry powder for subsequent use, which is easily re-dispersible in aqueous buffer. The yield of the polymeric nanoparticle was more than 90%. When MMA is replaced by other water insoluble vinyl derivatives like styrene (ST), the method of preparation remains the same, and the co-polymer does not change in its properties

### Example3

### Surface Modification of NIPAAM/VP/AA Copolymeric Micelles with 5 kD PEG Moiety

The formulations NVA631, NVA622 or NVA613 were prepared using the detailed protocol as described above. The exemplary functionalized PEG molecule used for post-copolymerization conjugation to AA was Methoxy-polyethylene glycol amine (Methoxy-PEGamine; molecular weight 5000 D). Conjugation of Methoxy-PEGamine with the carboxylic group of acrylic acid in the co-polymer was done by using EDCI as a crossslinker. Briefly, 100 mg of the lyophilized co-polymer powder was dissolved in 10 ml of phosphate buffer. To this, 5 mM of 1-ethyl-3-(3-dimethylaminopropyl)-carbodiimide hydrochloride (EDCI) was added and stirred for 30 minutes. Thereafter, 5 mg of Methoxy-PEGamine was added to the copolymer solution and stirred overnight at room temperature. The next day, the solution was dialyzed for 2-4 hrs to remove any unconjugated Methoxy-PEGamine using a 12 kD dialysis membrane followed by subsequent lyophilization. The resulting nanoparticles are designated as PEG-NVA631, PEG-NVA-622, and PEG-NVA613. Identical PEGylation can be performed with the NIPAAM/MMA/AA formulations, and are designated PEG-NMA631, PEG-NMA622, and PEG-NMA613, respectively.

### Example4

### Preparation of Polymeric Nanoparticles Encapsulating the Poorly Water Soluble Immunomodulatory and Anti-Cancer Drug, Rapamycin

The immunomodulatory and anti-cancer agent rapamycin is known to be poorly absorbed when administered through the oral route. To study the delivery of rapamycin using the nanoparticles of the invention, rapamycin was incorporated into NVA631, NVA622, NVA613, and NMA622 nanoparticles, or the respective PEGylated derivatives (PEG-NVA631, PEG-NVA622, PEG-NVA613 and PEG-NMA622) as follows: 100 mg of lyophilized dry powder of the respective nanoparticle was dispersed in 10 ml distilled water and was stirred well to reconstitute the micelles. The free drug rapamycin was dissolved in chloroform (10 mg/ml) and the drug solution in CHCl₃ was added to the polymeric solution slowly with constant vortexing and mild sonication. Rapamycin was directly loaded into the hydrophobic core of micelles. The drug-loaded micelles were then lyophilized to dry powder for subsequent use. Up to 3 mg of rapamycin per 100 mg of micellar powder was entrapped in each of the co-polymeric micelles (NVA631, NVA622, NVA613, and NMA622 and the respective PEGylated derivatives) to fond a drug loaded nanoparticle solution, thus giving a total loading of 3%(w/w) of the polymer.

This example shows that poorly water soluble drugs can be easily and efficiently loaded into the nanoparticles of the invention.

### Example5

### In Vivo Oral Administration of Polymeric Nanoparticles Encapsulating Rapamycin

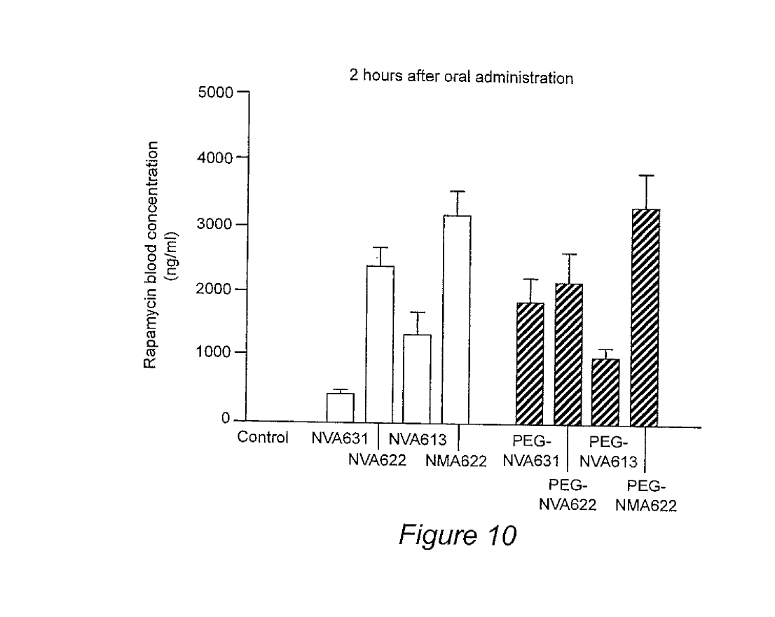

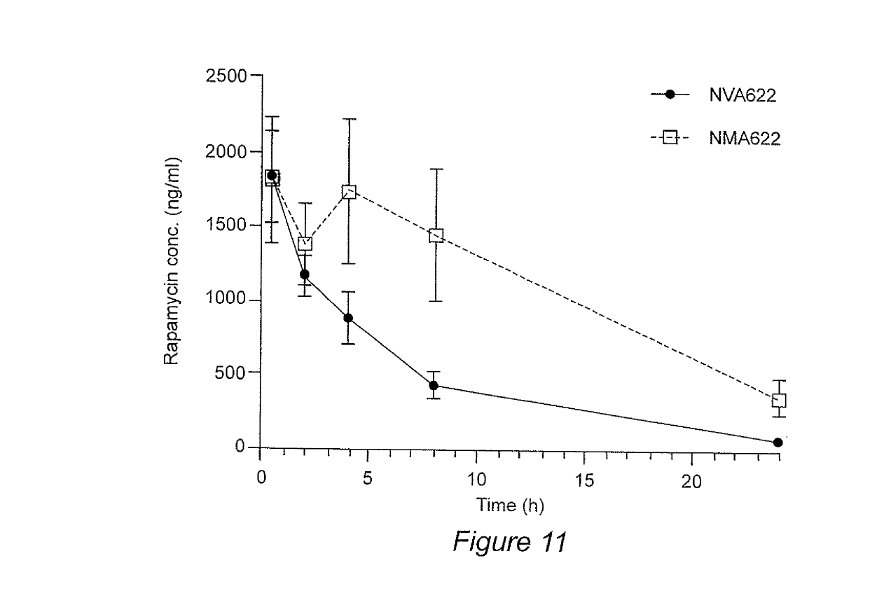

Rapamycin is a poorly water soluble drug that has low oral bioavailability. The objective of these experiments was to determine whether nano-encapsulation of rapamycin in the polymeric nanoparticles embodied in this invention can enhance absorption upon oral administration, compared to free rapamycin in aqueous media. Nine independent sets of C57B6 wild type mice (N=6 mice per set) were studied. Rapamycin was administered to the mice as oral free rapamycin (15 mg/kg body weight) suspended in water, or the equivalent amount of rapamycin encapsulated in NVA631, NVA622, NVA613 and NMA622 nanoparticles, or the respective surface modified PEGylated derivatives. All dosages were given by oral lavage. At 2 hours post oral administration, the mice were bled and rapamycin concentrations in the blood were determined by high performance liquid chromatography (HPLC). The results of this study are presented in FIG. 10. As can be seen, all nanoparticles tested successfully delivered high levels of rapamycin to the blood stream compared to free rapamycin in water, which was essentially undetectable. We ascribe these high systemic levels following oral delivery to both the nanoparticulate size (˜50 nm in diameter) of the carrier polymers, as well as their enhanced gastrointestinal mucoadhesivity due to the availability of free COO— (carboxyl) groups on the surface from the AA component in the polymer. Further, two of the nanoparticle formulations, NVA622 and NM622, had the highest two-hour blood levels, which we ascribe to an optimum molar ratio of mucoadhesive AA in the polymeric composition. This study also demonstrates that partial PEGylation of AA (as present in PEG-NVA631, PEG-NVA622, PEG-NVA613, and PEG-NMA622) does not impede the mucoadhesive tendencies of the nanoparticles, likely because a sufficient number of free COO— groups are available for mucosal adhesion even after the PEGylation. Therefore, the optional PEGylation of these nanoparticles, as sometimes required for long systemic circulation, does not impede oral bioavailability. The experiment in FIG. 11 confirms the rapid and robust oral uptake of the nanoparticle-encapsulated drug, with markedly high levels observed as early as 30 minutes after oral administration. Finally, the experiment in FIG. 12 confirms the equitable systemic distribution of the nanoparticle encapsulated drug in the circulation following their oral delivery, with near-identical levels of rapamycin observed in central and peripheral circulatory compartments. Thus, this example demonstrates the ability of polymeric nanoparticles embodied in this invention to efficiently deliver one or more encapsulated poorly water soluble drugs to the systemic circulation via the oral route.

### Example6

### In Vitro Growth Assays of Nanoparticle Formulation of an Anticancer Agent, and an Example of Combination Therapy Achieved Using Nanoparticle Formulations of Two Independent Anticancer Agents.

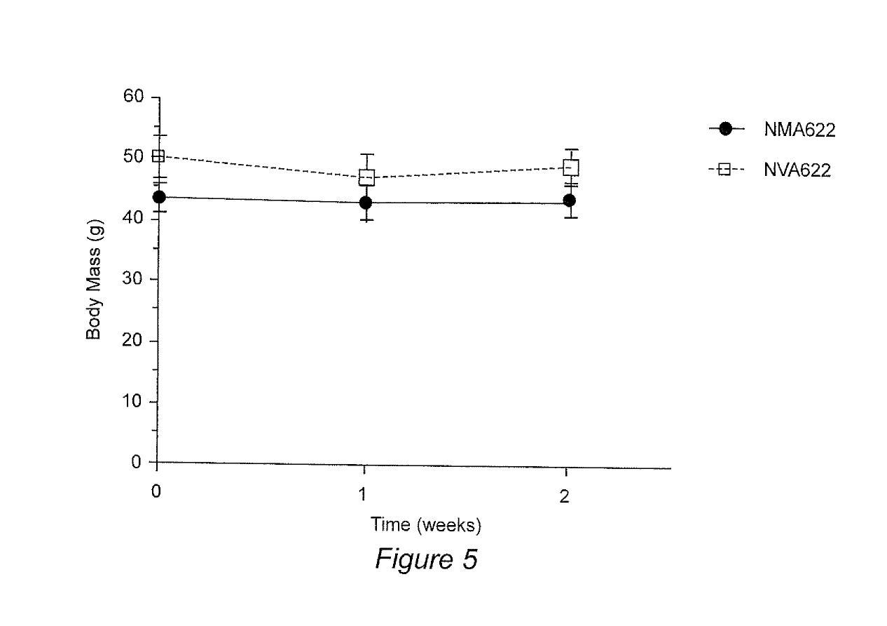

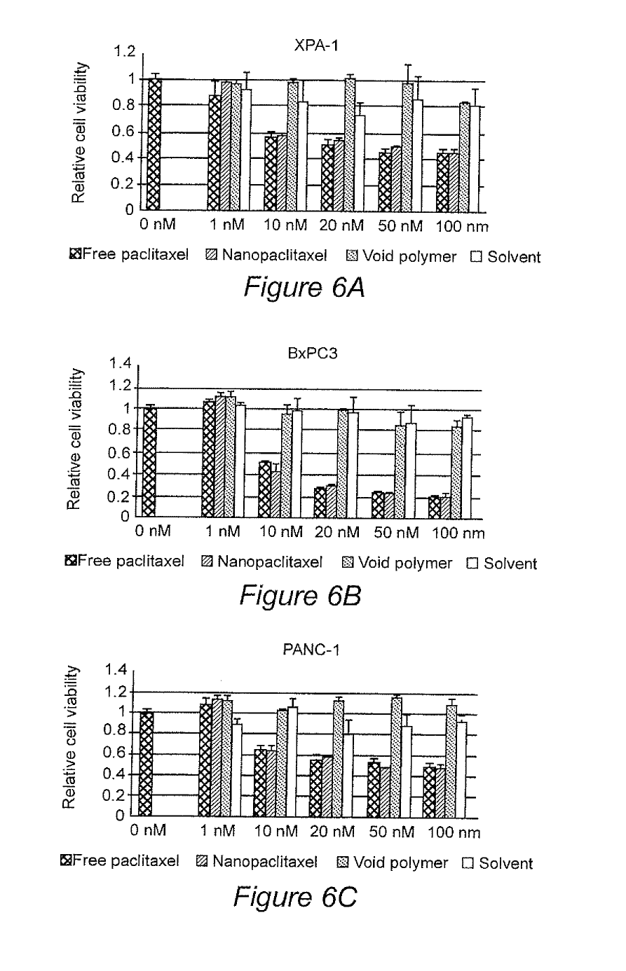

Paclitaxel is a poorly water soluble anticancer agent, and can be solubilized for dispersion in aqueous media using the polymeric nanoparticles described herein. Nanopaclitaxel encapsulated in NVA631 particles were utilized for in vitro cell viability (MTT) assays in a panel of three human pancreatic cancer cell lines (XPA-1, BxPC3, and PANG-1). The results of this study are presented in FIG. 6. As seen, the nanopaclitaxel demonstrates comparable potency to free drug for any given dose of paclitaxel, confirming that the process of nano-encapsulation does not diminish the activity of parent compound. The results of two independent therapeutic agents (nanopaclitaxel and nanocurcumin) are presented in FIG. 7. As seen, the combination of nanopaclitaxel and nanocurcumin demonstrates increased cytotoxicity than either free paclitaxel or nanopaclitaxel alone at any given dose of paclitaxel. Of note, and especially at the lower dosages used in two of the cell lines (XPA-1 and Panc-1), the combination of nanopaclitaxel and nanocurcumin also appears to have better efficacy than the combination of free paclitaxel and free curcumin, likely due to increased intracellular uptake of the nano-encapsulated compounds. At higher dosages, the combination therapy with either free or nano-encapsulated drugs appears to have comparable effects.

### Example7

### Surface Modification of Polymeric Nanoparticle Formulation by a Taste Masking Agent Aspartame, and Encapsulation of the Antifungal Agent Griseofulvin in the Surface Modified Nanoparticles

The antifungal agent griseofulvin is poorly water soluble, has poor oral bioavailability, and has a bitter taste that can affect patient compliance. In this example, we demonstrate the utility of “smart” polymeric nanoparticles (illustrative example is the composition NMA622) in being amenable to surface modification by taste masking agents, and the incorporation of griseofulvin within such modified nanoparticles. 10 ml of NMA 622 polymer nanoparticles dispersion (containing 100 mg of polymer) was mixed with 500 μl of 5 mM EDCI by stirring for complete dissolution. To the clear dispersion, 30 mg of solid Aspartame was added. The solution was stirred over night for 15 to 20 hours. The clear solution was then dialyzed through 12 kD cut off dialysis bag for 4 hours with change of external water at every one hour. To the dialyzed solution, 2 mg of solid griseofulvin was added, and the solution was sonicated for 30 mins for complete dispersion, followed by gentle heating with stirring at 50 to 60 C to achieve a clear solution. If required, the process of sonication followed by gentle heating with stirring was repeated till the solution was clear. The clear solution of nano-griseofulvin at room temperature was lyophilized to a dry powder for further use.

The release kinetics of griseofulvin from surface aspartame-conjugated polymeric nanoparticles at room temperature was further studied. 10 mg of lyophilized powder of griseofulvin loaded, surface modified NMA622 polymeric nanoparticles (designated “nano-griseofulvin”) were dissolved in 1 ml of water by vortexing. Then, 10 μl of the clear solution of nano-griseofulvin was added to 1 ml of water and the absorbance of the mixture was taken at 292 nm. After every two hours, the original nano-griseofulvin solution was centrifuged at 2000 rpm for 10 mins, and 10 μl of the centrifugate was pipetted carefully from the surface and was added to 1 ml of water. Absorbance was taken at 292 nm. After 10 hours, the original nano-griseofulvin solution was kept over night, and the 292 nm absorbance at 24 hours was measured, as described above. The absorbance was similarly measured at 48 and 72 hours. The % of release was calculated from the equation (Do−Dt)/Do×100 where Do is the absorbance at zero hours and Dt is the absorbance at t hours. In this calculation it is assumed that practically all the griseofulvin released from the nanoparticles settles down during centrifugation and that the concentration of griseofulvin in water is practically zero.

### Results:

| Time | OD | % release |
|---|---|---|
| 0 hr | 0.093 | 0.0 |
| 2 hrs | 0.085 | 8.6 |
| 4 hrs | 0.076 | 18.3 |
| 6 hrs | 0.072 | 23.0 |
| 10 hrs | 0.061 | 34.4 |
| 24 hrs | 0.053 | 43.0 |
| 48 hrs | 0.048 | 48.4 |
| 72 hrs | 0.018 | 80.6 |

This example demonstrates the encapsulation of another poorly water soluble drug, the antifungal agent griseofulvin, in the said polymeric nanoparticles, and the ability to alter the innate taste of the encapsulated medicament by taste masking agents conjugated to the nanoparticle surface. This example also demonstrates the favorable release kinetics of the nanoparticle-loaded drug over 72 hours, including absence of any “burst release” effects.

### Example8

### Conjugation of Water Soluble Anticancer Drug Gemcitabine on the Surface of Polymeric Nanoparticles and the Application of said “Nano-Gemcitabine” Preparation to in Vitro Cell Viability Assays in Human Cancer Cell Lines

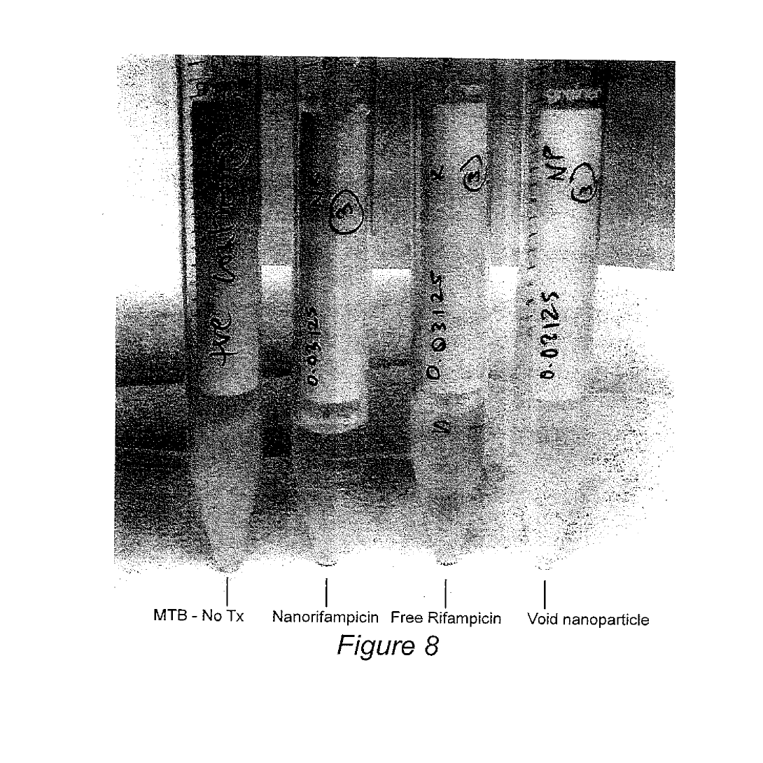

Gemcitabine is a water soluble compound, and thus differs from the poorly water soluble drugs discussed above that are encapsulated within the hydrophobic core of the polymeric nanoparticles. Herein, we describe the chemical conjugation of gemcitabine, as one illustrative example of water soluble drugs, to the hydrophilic surface of the polymeric nanoparticles. Such conjugation is expected to render gemcitabine amenable to oral delivery, utilizing the oral bioavailability properties of the said polymeric nanoparticles used as a carrier. 10 mg of NMA622 polymeric nanoparticles were dispersed in 10 ml of water by vortexing. To the clear solution, 6.5 mg of EDCI was added and was stirred for 10 mins. Thereafter, 10.2 mg of gemcitabine powder was added, while stirring was continued. The solution was stirred further for 15-20 hours. The clear solution was then dialysed for 3 hours through 121(D dialysis membrane against water. It was then lyophilized to dry powder for further use. In order to demonstrate retained anti-cancer effects of gemcitabine conjugated to polymeric nanoparticles, cell viability (MTT) assays were done as described in example 6, using the human pancreatic cancer cell line BxPC3. FIG. 9 confirms that nano-gemcitabine has comparable potency to free gemcitabine at 96 hours.

### Example9

### A Composite Polymer Nanoparticle Overcomes Multi-Drug Resistance and Ameliorates Doxorubicine Associated Cardiomyopathy

### Introduction

Resistance to chemotherapeutic drugs is a major impediment to a successful chemotherapeutic regimen. Cancer cells acquire drug resistance through a variety of mechanisms, not all of which are fully understood. Examples include host and tumor genetic alteration, epigenetic changes, changes in the tumor microenvironment, modification of the drug's cellular target, or blocking the drug's entry into the cell (1, 2). Single drug resistant cells are often cross-resistant to other structurally and functionally different drugs, a phenomenon known as multidrug resistance (MDR) (3). One key cause of acquired multidrug resistance is through energy-dependent efflux of cytotoxic agents through any of a 48-member family of ATP-binding cassette (ABC) transporters (2, 4, 5). Such transmembrane efflux pumps, including MDR1 and MRP1, aid in tumor cell survival by actively removing chemotherapeutic agents from the cell's cytoplasm.

Resistance to chemotherapeutic drugs such as anthracyclins, vinca alkaloids, RNA-transporter inhibitors, and microtubule-stabilizing drugs can be associated with either single or multiple ABC transporters (6, 7). For instance, resistance of metastatic tumors to the anthracyclin doxorubicin (DOX) has been linked to overexpression of ABC transporters ABCB1 (MDR1/P-glycoprotein) (7), ABCC1 (MRP1) (8), ABCC2 (MRP2) (9, 10) and ABCG2 (MXR, BRCP) (11-13). Further hampering the utility of doxorubicin in such instances, severe side effects including cardio- and nephrotoxicity limit the maximum tolerable dose in patients. At a cumulative dose of 550 mg/m2 of DOX, 26% of patients develop congestive heart failure (CHF) (14), a condition that is lethal in approximately 50% of cases. The rate of CHF is further increased in pediatric patients, with the frequency of CHF in pediatric acute lymphoblastic leukemia (ALL) patients, for example, as high as 57% (15-17). In some instances, combinatorial treatments can help mitigate the cardiotoxicity of DOX to some extent. For instance, Phase II clinical trials have shown that Trastuzumab (Herceptin®, monoclonal antibody against human epidermal growth factor-2; HER2), when combined with doxorubicin, reduced cardiotoxicity in breast cancer patients (18, 19). Despite increased rates of survival with combination therapy, approximately 27% of patients still develop some fotin of cardiotoxicity (20), an alarmingly high rate.

Towards the goal of overcoming multidrug resistance, several synthetic small molecules and antibodies targeted against MDR proteins have been tested in vitro and in vivo (21-25); however, these inhibitors have largely failed in clinical trials due to toxicity and low serum stability (2). Natural products are gaining attention in MDR inhibition due to their low cytotoxicity profiles. Among the many naturally occurring MDR inhibitors, curcumin's role (extract of curcuma longa) in inhibiting multiple MDR pumps has been studied widely (26-33). As an added benefit, in addition to its MDR inhibition properties, curcumin also possesses potential cardioprotective effects (34). Despite its promise, treatments utilizing curcumin, either alone or in combination with chemotherapeutic drugs have not had great success in the clinic, primarily due to the poor bioavailability of free curcumin outside the tubular lower GI tract (35). We recently developed a polymeric nanoparticle formulation of curcumin (NanoCurc™) that significantly enhances the bioavailability of curcumin (36, 37). To investigate the effects of combination therapy using this highly bioavailable formulation of curcumin in conjunction with doxorubicin, we synthesized a doxorubicin-curcumin multidrug formulation we have called ‘NanoDoxCurc’ (NDC) (which is an exemplary curcumin-chemotherapeutic agent combination in LCST nanoparticles according to the present invention) by covalently grafting doxorubicin to the free carboxylic acid moiety of NVA622 polymer, and subsequently encapsulating curcumin in its inner shell. Experiments described here in show that curcumin encapsulated in a doxorubicin-anchored polymeric nanoparticle can overcome DOX resistance in a variety of MDR-overexpressing cell lines both in vitro as well as in vivo, significantly inhibiting the growth of DOX-resistant xenografts. Additionally, NanoDoxCurc shows no evidence of cardiotoxicity, overcoming the single greatest limitation of doxorubicin-based chemotherapy.

### Materials and Methods

Reagents: NVA622 polymer was purchased from Lakeshore Biomaterials. Doxorubicin and EDCI were purchased from Sigma-Aldrich. Curcumin was purchased from Sabinsa. Anti-MDR1, MRP1 and glutathione antibodies were procured from Santa Cruz Biotech. Anti-p65 and anti-MIB1 were purchased from Cell Signaling and Ventana Biological Systems, respectively.
Cells: Doxorubicin resistant clones, namely NCI/ADR, PC-3A, and RPMI8226/Dox, as well as parental cell lines, were cultured in RPMI 1640 medium supplemented with 10% FBS and pen/strep.
Synthesis of NanoDoxCurc (NDC): Doxorubicin was covalently grafted to the carboxylic acid residue of NVA622 polymer and curcumin was encapsulated within its inner shell. In brief, NVA622 polymer (200 mg) and EDCI (40 mg) were dissolved in distilled water (20 mL) and stirred for 30 min at room temperature. Doxorubicin (0.80 mg, 20 mg/mL in DMSO) was added to the reaction mixture and stirred for 6 h. The resulting reaction mixture was dialyzed for 12 h with exchange of fresh water every 2 h. The purified product ‘NanoDox’ (ND) was either lyophilized for use, or further processed to encapsulate curcumin as previously reported (37), and lyophilized to produce ‘NanoDoxCurc’ (NDC). The final concentration of drug was measured calorimetrically, and the concentrations of doxorubicin and curcumin were adjusted to 1.4 μg/mg and 10 μg/mg of polymer, respectively, for in vitro studies. ‘NanoCurc’ (NC) was synthesized as previously reported (15 μg/mg of polymer) (37). ND and NDC were reconstituted in cell culture medium at 10 mg/mL to yield 25 μM DOX and 290 μM curcumin. NC was resuspended at 7.5 mg/mL to yield 325 μM curcumin. For in vivo studies, drugs were reconstituted in sterile PBS, with ND and NDC loading at 2.5 μg/mg of polymer and 10 μg/mg of polymer for DOX and curcumin, respectively.
Trafficking of DOX into nucleus: Cells were seeded in 2-chambered slides one day prior to treatment. The next day either NDC or ND reconstituted in cell culture medium (500 μL/chamber, 10 mg/mL) were added to the appropriate chambers. After 2 h of treatment, medium was discarded, cells were fixed in 4% paraformaldehyde for 20 min, counterstained with DAPI, mounted, and examined using a confocal microscope (Zeiss) at 1000× final magnification.
Cell Survival Assay: A panel of three DOX-resistant cancer cell lines NCI/ADR (breast cancer), PC-3A (prostate cancer), and RPM18226/Dox (myeloma) were cultured in 96-well plates and treated with ND (10 mg/mL), NDC (10 mg/mL), and NC (7.5 mg/mL) for 2 h. Following treatment the plates were washed with PBS and the cells were cultured in fresh growth medium for a further 48 h. Growth inhibition was measured by CellTiter 96® Aqueous Cell Proliferation Assay (Promega) according to manufacturer's protocol.
Soft-Agar Assay: 1×104 cells were treated with ND, NDC, NC or medium alone for 2 h. Cells were washed and resuspended in 2 mL complete medium with 0.7% agar. This suspension was layered on solidified 2 mL base agar mixture of serum supplemented media and 1% agar on a 6-well plate. Subsequently, the plates were incubated at 37° C. with 5% CO2 for 14 days to allow for colony growth. The plates were then stained and colonies counted on ChemiDoc XRS instrument (Bio-Rad, Hercules, Calif.).
Rhodamine Exclusion Assay: Cells were seeded in a 6 well plate at 1.5×105 cells per well and cultured overnight. The next day, cultures were treated either with 6004 of cell culture medium or with 600 μL of reconstituted ND, NDC, or NC for 2 h. The cells were further incubated in fresh medium supplemented with 200 nM TMRM for 20 min. At the end of incubation, the cells were trypsinized, and suspended in PBS containing 20 mM EDTA and 2% FBS. The samples were analyzed in a BD FACSCalibur.
Western Blot: Cell pellets were lysed with RIPA buffer, and 50 μg of protein per sample was separated on a 4-20% SDS-polyacrylamide gradient gel (SDS-PAGE, Invitrogen). Proteins were transferred onto a nitrocellulose membrane (Hybond-ECL, Amersham Biosciences, NJ) and blocked for 1 h with 5% non-fat milk in PBS containing 0.5% Tween-20 (PBS-T). Blots were then incubated with appropriate primary antibody at 1:500 dilutions for 2 h at room temperature. After washing with PBS-T (3×10 mL, 5 min. each), the membrane was incubated with appropriate HRP-conjugated secondary antibody (Santa Cruz Biotech) at 1:5000 dilution for 60 min. After washing with PBS-T (3×10 mL, 5 min. each), chemiluminescence film was developed after addition of the ECL substrate. Anti-actin antibody (dilution of 1:2000) was used as an internal control for protein loading.
Xenograft Studies: Flanks of 5-6 week old male athymic nu/nu mice (Harlan Laboratories, Indianapolis, Ind.) were injected with 5×106 PC-3A or RPMI8226/Dox cells suspended in a total volume of 200 μL [PBS/Matrigel (BD Biosciences), 1:1 (v/v), pre-chilled to 4° C.]. One week after the injection of tumor cells, when palpable subcutaneous tumors were present, twenty mice per tumor type with successfully engrafted xenografts were randomized into four cohorts of five animals each and administered intraperitoneally with (i) ND at a dose of 6 mg/kg DOX equivalent, (ii) NDC at a dose of 6 mg/kg DOX equivalent and 24 mg/kg curcumin equivalent, (iii) NC at a dose of 30 mg/kg curcumin equivalent, and (iv) vehicle. Mice were monitored daily for any signs of toxicity or behavioral abnormalities. Tumor size and body weight were measured once every week. At the culmination of treatment, visceral organs and tumor tissues were harvested and either preserved in 10% neutral buffered formalin for histology and immunohistochemical studies, or snap frozen for further analysis.
P388/Dox Ascites: P388/Dox DOX-resistant ascites were purchased from NCI (Fredrick, Md.), and were implanted intraperitoneally in two BDF1 mice (6 weeks, Harlan Laboratories, Indianapolis, Ind.). After 7 days ascitic fluid was collected via syringe and injected into 24 BDF1 mice. The following day mice were randomized into three arms receiving daily either (i) ND at a dose of 6 mg/kg DOX equivalent, (ii) NDC at a dose of 6 mg/kg DOX equivalent and 24 mg/kg curcumin equivalent, and (iii) vehicle. After 6 days of treatment (following the first death in the vehicle arm) treatment was terminated and mice followed for survival for the remainder of the study.
Mouse Echo Cardiogram: 4-5 week old C57BL/6J mice (Harlan Laboratories, Indianapolis, Ind.) were injected intravenously with free doxorubicin, Doxil, ND, NDC and PBS buffer at 9 mg/kg doxorubicin equivalent once weekly for 4 weeks. One week following the last injection echo cardiogram was performed. All measurements were performed using the leading-edge method, as recommended by the American Society of Echocardiography (38). To perform echocardiography on conscious animals (39), mice were gently held in supine position in the palm of the hand. The left hemithorax was shaved and a 1-2 mm thick layer of prewarmed hypoallergenic ultrasonic transmission gel (Parker Laboratories, Fairfield, N.J.) was applied to the thorax. Transthoracic echocardiography was performed using a Hewlett-Packard Sono 5500 ultrasound machine with a 15 MHz transducer. Images were stored on a 1.2 GB magnetic optical disk (Hewlett Packard) and T120 VHS tape. Two-dimensional and left ventricle M-mode measurements were taken in two separate 3-4 min sessions. The heart was first imaged in two-dimensional mode in the parasternal short axis view at a sweep speed of 150 mm/s. From this mode, an M-mode cursor was positioned perpendicular to the inter-ventricular septum and the left ventricular posterior wall thickness (LVPW) at the level of the papillary muscles. From the M-mode, the left ventricular wall thickness and chamber dimensions were measured. For each mouse, three to five values for each measurement were obtained and averaged for evaluation. Two research technologists trained in cardiac echocardiography and blinded to the experimental groups performed the studies. Left ventricular end-diastolic dimension (LVEDD), left ventricular end-systolic dimension (LVESD), interventricular septal wall thickness at end diastole (IVSD), and LVPW thickness at end diastole (LVPWTED) were measured from the M-mode tracing. LV fractional shortening (FS), the percent change in left ventricle cavity dimensions, was calculated using the following equation: fractional shortening (%)=[(LVEDD−LVESD)/LVEDD]×100. Ejection fraction (EF) represents stroke volume as a percentage of end diastolic LV volume and was calculated from the following equation: ejection fraction (%)=[(LVEDD2−LVESD2)/LVEDD2]×100. The heart rate was determined by counting the diastole and systole cycles during M-mode imaging within a defined time interval and multiplying by the correction factor to obtain heartbeats per min.
Histology: Cardiac histopathology was assessed in each treatment group based on the method of Billingham (40) as modified by Gabrielson et al. (41). The hearts were fixed in 10% phosphate-buffered formalin, embedded in paraffin, sectioned at a thickness of 3 μm, and stained with toluidine blue. The frequency and severity of myocardial lesions induced by doxorubicin was assessed by light microscopic examination. The changes were graded on the basis of the number of cardiomyocytes showing necrosis, mineralization, and cytoplasmic vacuolization.
Immunohistology: Immunohistochemistry was performed on formalin-fixed paraffin-embedded tissue, using common lab techniques. Briefly, the slides were deparaffinized using xylenes and hydrated by a graded series of ethanol washes. Antigen retrieval was accomplished by heating the slides in citrate buffer (pH 6.0) at 90° C. for 20 minutes. Endogenous peroxidase activity was quenched by 10 min incubation in 3% H2O2, and nonspecific binding was blocked by incubation in 10% fetal bovine serum solution (Invitrogen, Carlsbad, Calif.) before incubation with the primary antibody. Chromogenic detection was enabled using the PowerVision+ Poly-HRP IHC kit (Immunovision Technologies, Norwell, Mass.) following the manufacturer's protocol. Slides were counterstained with Harris-hematoxylin solution. Primary antibodies utilized were: anti-p65 (dilution 1:200), anti-MIB-1 (Ki-67) (dilution 1:100). Quantification of signal was performed by evaluating 10 random high power fields (40× magnification) on each slide, and counting the total number of cells with positive labeling. In the case of Ki-67, only nuclear localization of chromogenic signal was counted as positive. Four independent xenografts were evaluated for each treatment condition. Fluorometric TUNEL assay was performed according to manufacturer's protocol (Promega).

### Results

### NDC Formulation Potentiates Nuclear Trafficking of Doxorubicin:

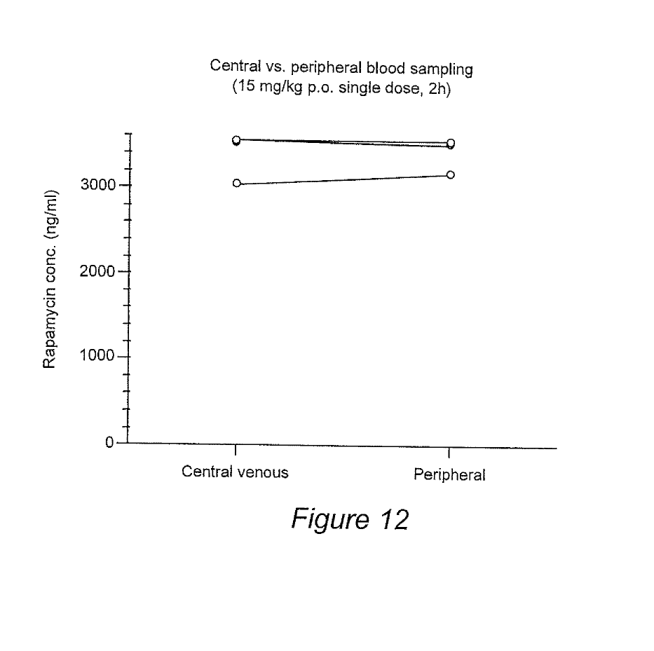

A doxorubicin-curcumin poly-pill was synthesized by covalently grafting doxorubicin to the carboxylic acid moiety NVA622 polymer (ND; FIG. 13) and by encapsulating curcumin to its hydrophobic core (NanoDoxCurc; NDC; FIG. 13). As with NanoCurc (NC) alone, this multi-drug NDC formulation was expected to increase the bioavailability of curcumin, subsequently inhibiting MDR protein function and allowing the chemotherapeutic drug doxorubicin to accumulate in the cell and to be trafficked to the nucleus.

To test the ability of NDC to overcome MDR, we chose three DOX resistant cell lines (NCI/ADR, PC-3A and RPMI8226/Dox) and their respective parental cell lines (PC-3, RPMI8226) for evaluation. MDR1, one of the most commonly over-expressed MDR-associated proteins in cancer (2), is abundantly expressed in NCI/ADR and RPMI8226/Dox cell lines; however, expression is absent in the parental RPM18226 line. Another key drug resistance marker in cancer, MRP1, was found to be expressed in PC-3A and RPMI8226/Dox cell lines with both parental lines lacking expression.

As an initial test of our hypothesis, we evaluated whether the curcumin-containing formulation NDC allowed accumulation of doxorubicin inside the nucleus. Cells were incubated with either NanoDox or NanoDoxCurc at a dose of 25 uM DOX equivalent for 2 h. The drug was subsequently washed off and nuclei stained with DAPI. In parental, non-DOX resistant cell lines ND colocalized with DAPI as expected (42), indicating accumulation of ND inside the nucleus. When resistant NCI/ADR, PC-3a, and RPMI8226/Dox cell lines were treated with ND, very little nuclear accumulation was observed. In stark contrast, treatment with NDC dramatically induced nuclear accumulation in DOX resistant cell lines, indicating the ability of cotreatment with a highly bioavailable curcumin to promote nuclear uptake of DOX.

To quantitatively confirm the ability of curcumin to reduce drug resistance by inhibiting drug effusion, we evaluated the exclusion of rhodamine dye by flow cytometry in MDR1 expressing RPMI8226/Dox and MRP1 expressing PC-3A cell lines. It was observed in untreated controls that MDR pumps can very efficiently efflux rhodamine dye out of the cytoplasm. In both cell lines, treatment with NC alone resulted in enhanced rhodamine accumulation; however, an even stronger effect could be observed upon treatment with NDC. Enhanced dye accumulation indicated the potential of curcumin to overcome ABC transporter function in MDR cell lines.

### NDC Significantly Reduces Viability and Clonogenicity of MDR-Overexpressing Cells.

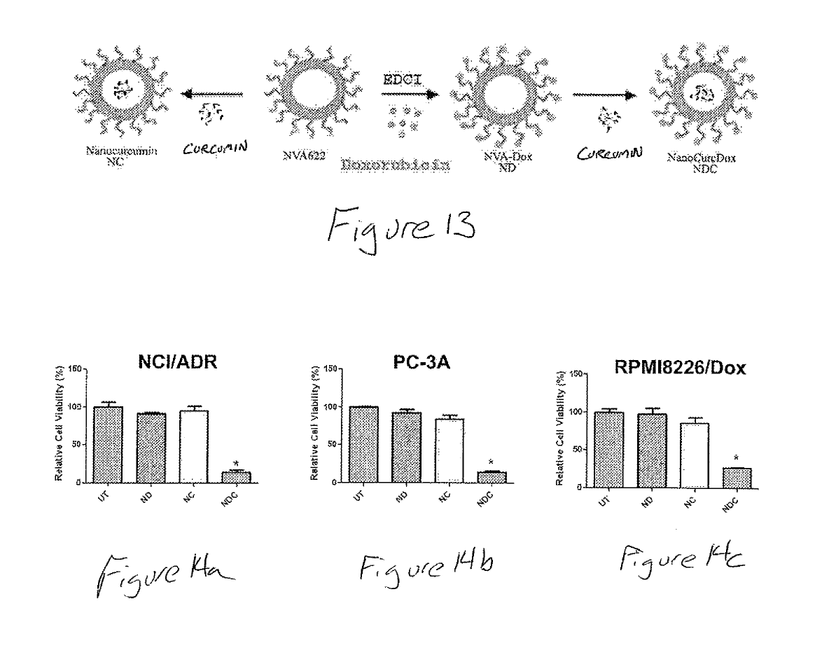

To test whether the NDC formulation increases the cytotoxic effects of DOX in DOX-resistant clones, we evaluated cell viability following treatment with ND, NC and NDC for 48 hours. All three lines were nearly completely refractory to ND alone, and only mild sensitivity to NC was observed in PC-3A and RPMI8226/Dox. In contrast, NDC treatment resulted in significant decreases in proliferation in all three DOX-resistant cell lines (FIGS. 14a-c). In a similar fashion, treatment with NDC significantly reduced clonogenicity, with ND alone showing only mild to moderate decreases in colony count. Interestingly, NC alone showed greater potency than ND in all three DOX-resistant cell lines.

### NDC Significantly Inhibits Growth of MDR-Overexpressing Xenografts in Vivo.

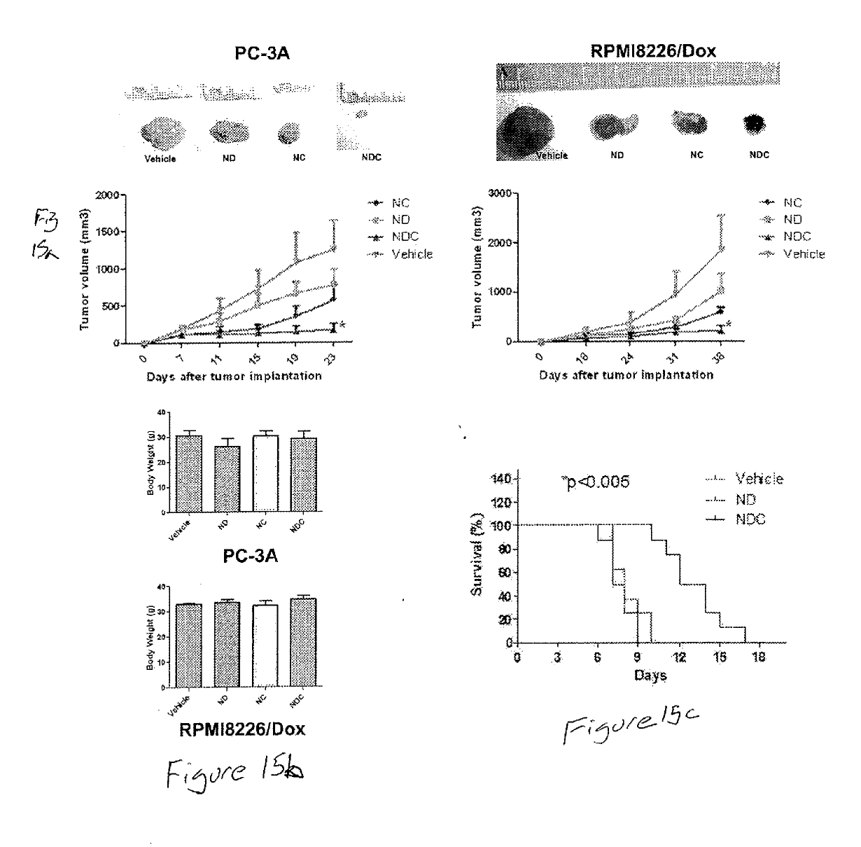

PC-3A and RPMI8226/Dox DOX-resistant clones were implanted subcutaneously in the right flank of athymic nude mice, and treated with either ND, NC, or NDC. In vivo nuclear accumulation of DOX was measured in formalin-fixed paraffin-embedded RPMI8226/Dox xenograft sections. ND treated xenografts clearly showed efflux of ND from the cells, with extracellular accumulation of ND; however, in NDC treated xenographs clear intracellular accumulation was visible. The presence of DOX was observed in the cytoplasmic compartment in ND-treated xenografts; however, dramatic nuclear accumulation of DOX was only observed in sections from NDC-treated tumors, indicating a clear benefit of co-treatment with curcumin. In both xenograft models, treatment with either ND or NC alone significantly reduced the rate of growth of tumor by approximately 50%. Demonstrating the benefit of the dual foitnulation, treatment with NDC yielded a greater than 90% reduction in tumor growth (FIG. 15a). Importantly, the body weight of animals treated with NDC for 2-3 weeks was not significantly different as compared to controls, suggesting a favorable toxicity profile at therapeutically relevant doses (FIG. 15b). Histological analysis of sections from treated tumors in both models showed significant necrotic regions in NDC-treated tumors, and to a lesser extent in NC-treated cases. Additionally, markedly lower proliferation was observed in RPMI8226/Dox xenografts treated with NDC as compared to ND, NC, or untreated control by staining for the cell proliferation marker Ki67. Additionally, immunofluorescence and western blot analysis of RPMI8226/Dox xenografts indicated greatly reduced expression of MDR1 in NC- and NDC-treated xenografts, suggesting curcumin-mediated inhibition of MDR1 as a factor in increased sensitivity to doxorubicin.

We also evaluated whether NDC increases the survival of wild-type BDF1 mice injected intraperitoneally with P388/ADR DOX-resistant ascites. Mice were treated with either NDC, ND, or vehicle for their complete lifespan following formation of ascites. Treatment with ND showed no survival benefit over vehicle controls; however, a significant survival increase of approximately 50% was observed upon treatment with NDC (FIG. 15c), again indicating the ability of curcumin co-treatment to overcome DOX resistance in vivo.

### NDC Exerts Substantially Reduced Cardiotoxicity as Compared to Doxorubicin and Doxil.

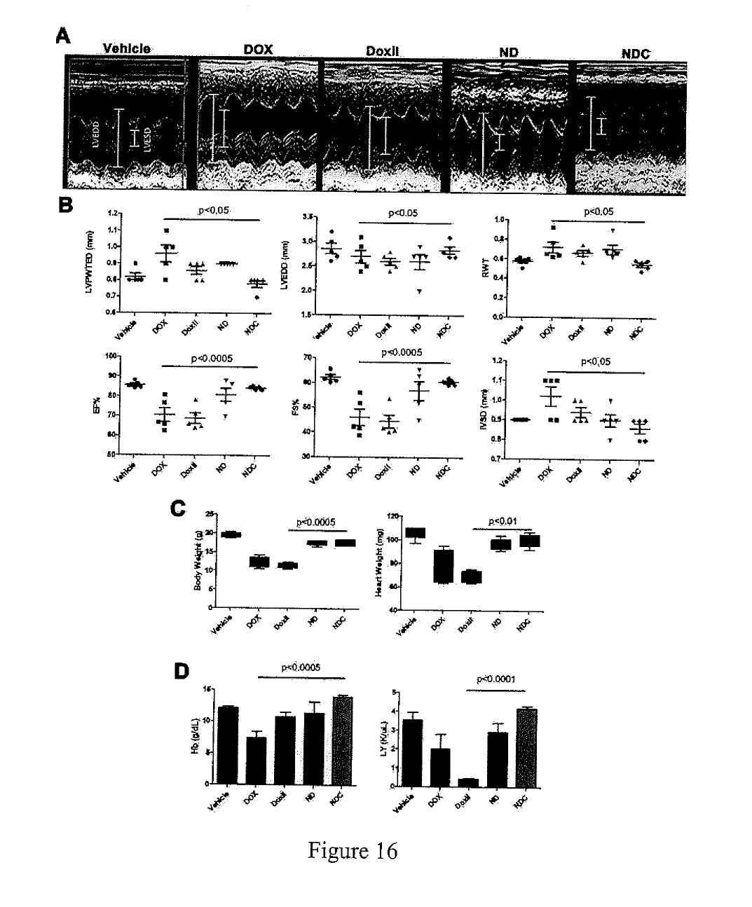

Increased cardiotoxicity is a leading concern in doxorubicin therapy, with cumulative dose limited to minimize cardiac damage. We compared the toxicity of the NDC formulation with that of free doxorubicin and Doxil, a commercially available liposomal formulation of DOX. C57BL/6 wild-type mice were injected intravenously with either buffered saline, free doxorubicin, Doxil, ND, or NDC once every week for 4 weeks (9 mg/kg DOX equivalent). One week after the last dose cardiac function of the mice was measured by echocardiogram (FIG. 16a). DOX and Doxil treated mice showed a significant increase in left ventricular end systolic dimension (LVESD), interventricular septal wall thickness at end diastole (IVSD), left ventricular posterior wall thickness at end diastole (LVPWTD); and hence a decrease of fractional shortening (FS), and ejection fraction (EF) was observed, indicating acute cardiomyopathy in these mice. ND-treated mice, and in particular NDC-treated mice, showed significantly reduced signs of cardiotoxicity (FIG. 16b).

Both DOX and Doxil treatment dramatically reduced mouse body weight and heart weight by more than 40% (FIG. 16c). As with other indicators of toxicity, ND- and NDC-treated mice showed no significant change in body and heart weight relative to controls. Blood samples were collected from experimental animals by cardiac puncture and hearts were collected for histological and molecular studies. Hemoglobin (Hb) levels dropped from an average of 12.5 g/dL in control mice to an average of 7.5 g/dL in DOX-treated mice, and lymphocyte count was significantly reduced in Doxil treated animals, indicating both anemia and severe lymphocytopenia (FIG. 16d). Interestingly, all hematological parameters in ND-and NDC-treated mice were unchanged as compared to vehicle controls, further demonstrating the reduced toxicity of these formulations.

Histological assays and TUNEL staining were also performed on heart cryosections to examine for indications of doxorubicin-induced apoptosis and cardiomyopathy. Toluidine blue-stained heart sections from DOX and Doxil treated animals presented widespread lesions indicated the occurrence of cardiomyopathy in these groups. In contrast, sections from ND- and NDC-treated mice were indistinguishable from those of vehicle-treated controls. Additionally, analysis of H&E stained sections revealed the presence of hypertrophic cardiac cells—characterized by elongated nuclei—in both DOX- and Doxil-treated mice; however, no such lesions were found in sections from ND and NDC groups. TUNEL staining indicated widespread apoptosis in cardiac cells in both DOX- and Doxil-treated mice. In contrast, few apoptotic cells were observed in ND treated mice, and no apoptotic cells were observed in NDC and vehicle treated groups.

To evaluate the degree of oxidative stress in treated mice, levels of the antioxidants glutathione and glutathione peroxidase (GPx) were evaluated by western blot and ELISA, respectively, in mouse heart lysates. As expected, there was a marked decrease in both glutathione and GPx levels in both DOX- and Doxil-treated groups; however, both ND- and NDC-treated groups showed similar levels to those of vehicle controls, indicating much lower levels of oxidative stress in ND- and NDC-treated mice. Taken together, these results suggest that both the polymeric formulation and the presence of curcumin each provide a distinct level of enhanced cardioprotection compared to either DOX or Doxil.

### Discussion

Multiple drug resistance caused by overexpression of ATP-binding cassette (ABC) transporters is a major impediment in cancer chemotherapy (2). Current approaches to overcome MDR include a focus on drug discovery, with, in many cases, an end goal of combination therapy (2). Although curcumin has been extensively studied as an inhibitor of ABC-transporter function, its use in vivo and in the clinic has been severely limited by the poor bioavailability of this highly hydrophobic small molecule. Following our recent development of a highly-bioavailable nanoparticle-encapsulated formulation of curcumin (NanoCurc™) (36, 37), this Example describes the development of a composite curcumin-doxorubicin nanoparticle, NanoDoxCurc (NDC), which will overcome MDR protein function and potentially provide lasting therapy for patients in an important step forward in improving overall cancer survival. As an additional benefit, curcumin, a natural antioxidant, was expected to reduce cardiac toxicity in such a composite nanoparticle, opening the possibility of increased safety at higher cumulative doses of doxorubicin.

Following the synthesis of NanoDoxCurc by covalent modification of the existing NanoCurc formulation, we began investigating the in vitro effects of this new formulation. We observed that curcumin strongly represses the MDR phenotype in DOX-resistant cancer cell lines that constitutively overexpress the MDR proteins MDR1 and MRP1. In the context of rhodamine exclusion—a standard assay to assess MDR function (43)—we observed a significant increase in dye uptake in MDR-overexpressing cells treated with the curcumin-containing formulations NDC and NC. This clear improvement in drug uptake and retention supports our hypothesis that the presence of curcumin in the NDC formulation inhibits MDR-dependent drug efflux. Similarly, we observed that the addition of curcumin with NDC completely abrogated the DOX nuclear exclusion pattern characteristic of MDR cells that was observed in ND treated DOX-resistant cells (42). This inhibition of the MDR phenotype by NDC was accompanied by significant synergistic decreases in both cell proliferation (FIGS. 14a-c) and soft agar colony formation, indicating the potential therapeutic relevance.

Based on such successful in vitro data, we tested the efficacy of the NDC formulation in vivo using MDR-overexpressing DOX-resistant xenografts (FIGS. 15a-c). NDC inhibited tumor growth significantly as compared to vehicle, NC, or ND alone in PC-3A and RPMI8226/Dox xenografts, yet mice showed no signs of toxicity, maintaining full body weight and demonstrating no overt behavioral changes throughout the duration of treatment. Interestingly, while both ND and NC each showed a degree of tumor growth inhibition, the composite nanoparticle NDC showed nearly complete growth inhibition over the duration of the study. Similar DOX uptake patterns to those observed in vitro were found in ND- and NDC-treated xenografts, and subsequent expression studies as measured by western blot and immunofluorescence on excised xenografts showed markedly decreased levels of MDR1 in NC and NDC treated tumors. To further evaluate the therapeutic efficacy of NDC, we utilized BDF1 wild-type mice with MDR-overexpressing P388 DOX-resistant ascites (44), chosen for the increased translational relevance of utilizing a syngenic model of leukemia. Treatment with NDC markedly increased the median survival by more than 50% as compared to ND or vehicle treatment (FIG. 15c). Taken together these results demonstrate the ability of nanoparticle-delivered curcumin to effectively overcome MDR in vivo by inhibiting ABC-transporter expression, restoring the excellent therapeutic efficacy of doxorubicin in a variety of model systems.

In the treatment of malignancies with DOX, the occurrence of cardiotoxicity is dose-dependent, limiting the cumulative dose a patient may receive, and thus limiting the therapeutic efficacy of the drug (45). Because the mechanism of DOX-induced cardiotoxicity is independent of its mechanism of action (20), there exists the potential to selectively block the toxicity of DOX without affecting its therapeutic benefit. Since curcumin has been suggested to ameliorate DOX-induced cardiomyopathy, we expected that a composite nanoparticle formulation of DOX and curcumin (NDC) would comprise a highly effective means of combination therapy. To evaluate this hypothesis, we investigated the cardiotoxicity of NDC in a model of high cumulative dose toxicity in C57BL6/J wild-type mice as evaluated by echocardiogram. As expected, mice treated with Dox and Doxil showed clear signs of cardiotoxicity. In particular significant decreases in both ejection fraction (EF) and fractional shortening (FS) were observed, key clinical indicators of impaired myocardial function (15, 45). In stark contrast, mice treated with ND or NDC showed significantly reduced impairment of cardiac function. Hemoglobin and leukocyte counts were also found to be reduced by DOX and Doxil; however, both ND and NDC treated mice showed counts similar to controls, indicating significant reductions in hematological toxicity as well.

Histological staining showed the presence of doxorubicin and Doxil induced cardiomyopathy and hypertrophy in cardiac cells, both of which were largely absent in ND and NDC treated groups. TUNEL assays performed on cardiac sections indicated that DOX and Doxil also induced apoptosis of cardiac cells. As the major mechanism of doxorubicin-induced cardiotoxicity is oxidative stress (20, 45), we evaluated glutathione levels and glutathione peroxidase activity in cardiac tissue. Reduced glutathione expression and glutathione peroxidase activity were observed in cardiac tissue of DOX and Doxil treated mice, indicating that both treatments induce oxidative stress upon cardiac tissue. Both ND and NDC treated mice showed high levels of glutathione, while only NDC showed elevated glutathione peroxidase activity, both indicating lower levels of oxidative stress. This, in combination with the overall enhanced cardioprotection of ND versus DOX and Doxil, indicates that the observed abrogation of cardiotoxicity in NDC is two-fold. First, the nanoparticle formulation ND itself provides a level of cardioprotection from doxorubicin not seen even in the liposomal formulation Doxil, while still maintaining efficacy. Second, the addition of curcumin in the form of a composite nanoparticle provides an additional level of cardioprotection, likely through a decrease in oxidative stress, as indicated by glutathione peroxidase levels.

In conclusion, this Example shows that we designed an exemplary composite polymeric nanoparticle, which has doxrorubicin covalently bound to the surface of the nanoparticle, and curcumin encapsulated within its inner core. This composite nanoparticle (NDC) can unequivocally overcome multidrug resistance as demonstrated by monitoring expression of MDR proteins and drug uptake, which translates into significant improvements in in vivo efficacy against DOX-resistant xenografts and syngenic ascites. Additionally, NDC shows significantly reduced cardiotoxicity in mice receiving high cumulative doses due to the cardioprotection afforded both by the nanoparticle itself, and by the encapsulated highly-bioavailable curcumin. Such composite nanoparticles have great promise for clinical translation, as they directly address multiple challenges by both overcoming resistance and enhancing safety, effectively ‘killing two birds with one stone.

### REFERENCES FOR EXAMPLE 9

1. Gone M E, Mohammed M, Ellwood K, Hsu N, Paquette R, Rao P N, et al. Clinical resistance to STI-571 cancer therapy caused by BCR-ABL gene mutation or amplification. Science. 2001; 293:876-80.

2. Szakacs G, Paterson J K, Ludwig J A, Booth-Genthe C, Gottesman M M. Targeting multidrug resistance in cancer. Nat Rev Drug Discov. 2006; 5:219-34.

3. Durand R E, Olive P L. Resistance of tumor cells to chemo- and radiotherapy modulated by the three-dimensional architecture of solid tumors and spheroids. Methods Cell Biol. 2001; 64:211-33.

4. Gottesman M M, Fojo T, Bates S E. Multidrug resistance in cancer: role of ATP-dependent transporters. Nature reviews Cancer. 2002; 2:48-58.

5. Higgins C F. ABC transporters: from microorganisms to man. Annu Rev Cell Biol. 1992; 8:67-113.

6. Dean M, Rzhetsky A, Allikmets R. The human ATP-binding cassette (ABC) transporter superfamily. Genome research. 2001; 11:1156-66.

7. Ambudkar S V, Dey S, Hrycyna C A, Ramachandra M, Pastan I, Gottesman M M. Biochemical, cellular, and pharmacological aspects of the multidrug transporter. Annu Rev Pharmacol Toxicol. 1999; 39:361-98.

8. Cole S P, Bhardwaj G, Gerlach J H, Mackie J E, Grant C E, Almquist K C, et al. Overexpression of a transporter gene in a multidrug-resistant human lung cancer cell line. Science. 1992; 258:1650-4.

9. Borst P, Evers R, Kool M, Wijnholds J. A family of drug transporters: the multidrug resistance-associated proteins. Journal of the National Cancer Institute. 2000; 92:1295-302.

10. Paulusma C C, Bosnia P J, Zaman G J, Bakker C T, Otter M, Scheffer G L, et al. Congenital jaundice in rats with a mutation in a multidrug resistance-associated protein gene. Science. 1996; 271:1126-8.

11. Litman T, Druley T E, Stein W D, Bates S E. From MDR to MXR: new understanding of multidrug resistance systems, their properties and clinical significance. Cell Mol Life Sci. 2001; 58:931-59.

12. Honjo Y, Hrycyna C A, Yan Q W, Medina-Perez W Y, Robey R W, van de Laar A, et al. Acquired mutations in the MXR/BCRP/ABCP gene alter substrate specificity in MXR/BCRP/ABCP-overexpressing cells. Cancer Research. 2001; 61:6635-9.

13. Doyle L A, Yang W, Abruzzo LV, Krogmann T, Gao Y, Rishi A K, et al. A multidrug resistance transporter from human MCF-7 breast cancer cells. Proceedings of the National Academy of Sciences of the United States of America. 1998; 95:15665-70.

14. Swain S M, Whaley F S, Ewer M S. Congestive heart failure in patients treated with doxorubicin: a retrospective analysis of three trials. Cancer. 2003; 97:2869-79.

15. Lipshultz S E, Lipsitz S R, Sallan S E, Dalton V M, Mone S M, Gelber R D, et al. Chronic progressive cardiac dysfunction years after doxorubicin therapy for childhood acute lymphoblastic leukemia. Journal of clinical oncology. 2005; 23:2629-36.

16. Lipshultz S E, Lipsitz S R, Mone S M, Goorin A M, Sallan S E, Sanders S P, et al. Female sex and drug dose as risk factors for late cardiotoxic effects of doxorubicin therapy for childhood cancer. The New England journal of medicine. 1995; 332:1738-43.

17. Lipshultz S E, Colan S D, Gelber R D, Perez-Atayde A R, Sallan S E, Sanders S P. Late cardiac effects of doxorubicin therapy for acute lymphoblastic leukemia in childhood. The New England journal of medicine. 1991; 324:808-15.

18. Slamon D J, Clark G M, Wong S G, Levin W J, Ullrich A, McGuire W L. Human breast cancer: correlation of relapse and survival with amplification of the HER-2/neu oncogene. Science. 1987; 235:177-82.

19. Slamon D J, Leyland-Jones B, Shak S, Fuchs H, Paton V, Bajamonde A, et al. Use of chemotherapy plus a monoclonal antibody against HER2 for metastatic breast cancer that overexpresses HER2. The New England journal of medicine. 2001; 344:783-92.

20. Chatterjee K, Zhang J Q, Honbo N, Karliner J S. Doxorubicin Cardiomyopathy. Cardiology. 2010; 115:155-62.

21. Friedenberg W R, Rue M, Blood E A, Dalton W S, Shustik C, Larson R A, et al. Phase W study of PSC-833 (valspodar) in combination with vincristine, doxorubicin, and dexamethasone (valspodarNAD) versus VAD alone in patients with recurring or refractory multiple myeloma (E1A95): a trial of the Eastern Cooperative Oncology Group. Cancer. 2006; 106:830-8.

22. Tan B, Piwnica-Worms D, Ratner L. Multidrug resistance transporters and modulation. Curr Opin Oncol. 2000; 12:450-8.

23. Tsuruo T, Iida H, Tsukagoshi S, Sakurai Y. Overcoming of vincristine resistance in P388 leukemia in vivo and in vitro through enhanced cytotoxicity of vincristine and vinblastine by verapamil. Cancer Research. 1981; 41:1967-72.

24. Wu C P, Calcagno A M, Ambudkar S V. Reversal of ABC drug transporter-mediated multidrug resistance in cancer cells: evaluation of current strategies. Curr Mol Pharmacol. 2008; 1:93-105.

25. Lee C H. Reversing agents for ATP-binding cassette (ABC) transporters: application in modulating multidrug resistance (MDR). Curr Med Chem Anticancer Agents. 2004; 4:43-52.

26. Venkatesan N. Curcumin attenuation of acute adriamycin myocardial toxicity in rats. British journal of pharmacology. 1998; 124:425-7.

27. Choi B H, Kim C G, Lim Y, Shin S Y, Lee Y H. Curcumin down-regulates the multidrug-resistance mdr1b gene by inhibiting the PI3K/Akt/NF kappa B pathway. Cancer Letters. 2008; 259:111-8.

28. Chearwae W, Wu C P, Chu H Y, Lee T R, Ambudkar S V, Limtrakul P. Curcuminoids purified from turmeric powder modulate the function of human multidrug resistance protein 1 (ABCC1). Cancer Chemotherapy and Pharmacology. 2006; 57:376-88.

29. Chearwae W, Shukla S, Limtrakul P, Ambudkar S V. Modulation of the function of the multidrug resistance-linked ATP-binding cassette transporter ABCG2 by the cancer chemopreventive agent curcumin. Molecular Cancer Therapeutics. 2006; 5:1995-2006.

30. Chearwae W, Anuchapreeda S, Nandigama K, Ambudkar S V, Limtrakul P. Biochemical mechanism of modulation of human P-glycoprotein (ABCB1) by curcumin I, H, and DT purified from Turmeric powder. Biochemical Pharmacology. 2004; 68:2043-52.

31. Anuchapreeda S, Leechanachai P, Smith M M, Ambudkar S V, Limtrakul P N. Modulation of P-glycoprotein expression and function by curcumin in multidrug-resistant human KB cells. Biochemical Pharmacology. 2002; 64:573-82.

32. Limtrakul P. Curcumin as chemosensitizer. Adv Exp Med Biol. 2007; 595:269-300.

33. Shukla S, Wu C P, Ambudkar S V. Development of inhibitors of ATP-binding cassette drug transporters: present status and challenges. Expert Opin Drug Metab Toxicol. 2008; 4:205-23.

34. Srivastava G, Mehta J L. Currying the heart: curcumin and cardioprotection. J Cardiovasc Pharmacol Ther. 2009; 14:22-7.

35. Sharma R A, McLelland H R, Hill K A, Ireson C R, Euden S A, Manson M M, et al. Pharmacodynamic and pharmacokinetic study of oral Curcuma extract in patients with colorectal cancer. Clinical cancer research: an official journal of the American Association for Cancer Research. 2001; 7:1894-900.

36. Bisht S, Feldmann G, Soni S, Ravi R, Karikar C, Maitra A, et al. Polymeric nanoparticle-encapsulated curcumin (“nanocurcumin”): a novel strategy for human cancer therapy. Journal of Nanobiotechnology. 2007; 5:3.

37. Bisht S, Mizuma M, Feldmann G, Ottenhof N A, Hong S-M, Pramanik D, et al. Systemic Administration of Polymeric Nanoparticle-Encapsulated Curcumin (NanoCurc) Blocks Tumor Growth and Metastases in Preclinical Models of Pancreatic Cancer. Molecular Cancer Therapeutics. 2010; 9:2255-64.

38. Sahn D J, DeMaria A, Kisslo J, Weyman A. Recommendations regarding quantitation in M-mode echocardiography: results of a survey of echocardiographic measurements. Circulation. 1978; 58:1072-83.

39. Yang X P, Liu Y H, Rhaleb N E, Kurihara N, Kim H E, Carretero O A. Echocardiographic assessment of cardiac function in conscious and anesthetized mice. Am J Physiol. 1999; 277:H1967-74.

40. Billingham M. Role of endomyocardial biopsy in diagnosis and treatment of heart disease. In: Silver M, editor. Cardiovascular Pathology. Ed. 2 ed. New York: Churchill Livingston; 1991. p. 1465-86.

41. Gabrielson K L, Hogue B A, Bohr V A, Cardounel A J, Nakajima W, Koller J, et al. Mitochondrial toxin 3-nitropropionic acid induces cardiac and neurotoxicity differentially in mice. The American journal of pathology. 2001; 159:1507-20.

42. Featherstone J M, Speers A G, Lwaleed B A, Hayes M C, Cooper A J, Birch B R. The nuclear membrane in multidrug resistance: microinjection of epirubicin into bladder cancer cell lines. BJU Int. 2005; 95:1091-8.

Lamy T, Drenou B, Grulois I, Fardel O, Jacquelinet C, Goasguen J, et al. Multi-drug resistance (MDR) activity in acute leukemia determined by rhodamine 123 efflux assay. Leukemia. 1995; 9:1549-55.

44. Ramachandran C, Sauerteig A, Sridhar K S, Thurer R J, Krishan A. MDR-1 gene expression, anthracycline retention and cytotoxicity in human lung-tumor cells from refractory patients. Cancer Chemotherapy and Pharmacology. 1993; 31:431-41.

45. Singal P K, Iliskovic N. Doxorubicin-induced cardiomyopathy. N Engl J Med. 1998; 339:900-5.

While the invention has been described in terms of its preferred embodiments, those skilled in the art will recognize that the invention can be practiced with modification within the spirit and scope of the appended claims.

What is claimed is:

1. A method for preparing polymeric nanoparticles having a lower critical solution temperature (LCST) above 37 C which are suitable for systemic administration to a subject for providing chemotherapy to said subject, comprising the steps of:
dissolving in aqueous fluid to form micelles from monomers consisting of
N-isopropylacrylamide (NIPAAM), acrylic acid (AA), and at least one vinyl monomer selected from the group consisting of vinyl acetate, 4-vinyl benzoic acid, methylmethacrylate, vinylmethacrylate, N-vinylpyrrolidone, N-vinyl piperidone, N-vinyl caprolacum, N-vinyl carbazole, and styrene,
wherein said NIPAAM, said AA, and said vinyl monomer are present at molar ratios of 50-70:10-30:10-30 for NIPAAM:AA:vinyl monomer;
polymerizing said micelles to form polymeric nanoparticles in solution;
removing unreacted materials from said solution after said polymerizing step; and
adding curcumin and one or more chemotherapeutic agents selected from the group consisting of anthracyclines, taxanes, chemotherapeutic platinum compounds, and topoisomerase inhibitors, and allowing said curcumin and said one or more chemotherapeutic agents to become entrapped within polymerized micelles in said solution or to become conjugated to the surface of said polymerized micelles in said solution, optionally functionalizing AA with polyethylene glycol (PEG) amine.

2. The method of claim 1 wherein said one or more chemotherapeutic agents include an anthracycline which is doxorubicin.

3. The method of claim 2 wherein said polymeric nanoparticles have a diameter of 50-100 nm or smaller.

4. The method of claim 3 wherein said curcumin is located within said polymeric nanoparticles and said doxorubicin is located on a surface of said polymeric nanoparticles.

5. The method of claim 1 wherein said polymeric nanoparticles have a diameter of 50-100 nm or smaller, and wherein said curcumin is located within said polymeric nanoparticles and said one or more chemotherapeutic agents are located on a surface of said polymeric nanoparticles.

6. The method of claim 1 wherein said adding step is performed before or during said polymerizing step.

7. The method of claim 1 wherein said adding step is performed after said polymerizing step.

8. The method of claim 1 wherein said polymerizing step is performed in the presence of an inert gas.

9. The method of claim 1 further comprising the step of functionalizing AA with PEG amine.

10. Polymeric nanoparticles having a lower critical solution temperature (LCST) above 37 C which are suitable for systemic administration to a subject for providing chemotherapy to said subject, comprising:
a polymeric substrate formed from monomers consisting of
N-isopropylacrylamide (NIPAAM), acrylic acid (AA), and at least one vinyl monomer selected from the group consisting of vinyl acetate, 4-vinyl benzoic acid, methylmethacrylate, vinylmethacrylate, N-vinylpyrrolidone, N-vinyl piperidone, N-vinyl caprolacum, N-vinyl carbazole, and styrene,
wherein said NIPAAM, said AA, and said vinyl monomer are present at molar ratios of 50-70:10-30:10-30 for NIPAAM:AA:vinyl monomer,
wherein said polymeric substrate is in the form of a particle having a diameter of 50-100 nm or smaller;
curcumin entrapped within said polymeric substrate; and
one or more chemotherapeutic agents associated with said polymeric substrate, said one or more chemotherapeutics agents being selected from the group consisting of anthracyclines, taxanes, chemotherapeutic platinum compounds, and topoisomerase inhibitors.

11. The polymeric nanoparticles of claim 10 wherein said one or more chemotherapeutic agents include doxorubicin, and wherein said doxorubicin is associated with an external surface of said polymeric substrate.

12. The polymeric nanoparticles of claim 10 wherein said one or more chemotherapeutic agents are associated with an external surface of said polymeric substrate.

13. An injectable formulation for systemic administration to a subject for providing chemotherapy to a subject comprising:
a carrier fluid; and
polymeric nanoparticles dispersed in said carrier fluid,
said polymeric nanoparticles having a lower critical solution temperature (LCST) above 37 C, said polymeric nanoparticles including
a polymeric substrate formed from monomers consisting of
N-isopropylacrylamide (NIPAAM), acrylic acid (AA), and at least one vinyl monomer selected from the group consisting of vinyl acetate, 4-vinyl benzoic acid, methylmethacrylate, vinylmethacrylate, N-vinylpyrrolidone, N-vinyl piperidone, N-vinyl caprolacum, N-vinyl carbazole, and styrene,
wherein said NIPAAM, said AA, and said vinyl monomer are present at molar ratios of 50-70:10-30:10-30 for NIPAAM:AA:vinyl monomer,
wherein said polymeric substrate is in the form of a particle having a diameter of 50-100 nm or smaller,
curcumin entrapped within said polymeric substrate, and
one or more chemotherapeutic agents associated with said polymeric substrate, said one or more chemotherapeutics agents being selected from the group consisting of anthracyclines, taxanes, chemotherapeutic platinum compounds, and topoisomerase inhibitors.

14. The injectable formulation of claim 13 wherein said one or more chemotherapeutic agents include doxorubicin, and wherein said doxorubicin is associated with an external surface of said polymeric substrate.

15. The injectable formulation of claim 13 wherein said one or more chemotherapeutic agents are associated with an external surface of said polymeric substrate.
